(19) United States
(12) Patent Application Publication
(10) Pub. No.: US 2024/0277826 A1
(43) Pub. Date: Aug. 22, 2024

(54) PHARMACEUTICAL COMPOSITION FOR TREATMENT OF COVID-19 AND RELATED PATHOLOGIES

(71) Applicant: UNTHREADT B.V., Amsterdam (NL)

(72) Inventors: Nisar Ahmed KHAN, Amsterdam (NL); Robbert BENNER, Barendrecht (NL)

(21) Appl. No.: 18/003,036

(22) PCT Filed: Jun. 20, 2021
(86) PCT No.: PCT/NL2021/050388

§ 371 Date: Dec. 22, 2022

(30) Foreign Application Priority Data
Jun. 24, 2020 (EP) 20182108.9

(51) Int. Cl. A61K 39/12; A61P 31/14

(52) U.S. Cl. CPC A61K 39/12; A61P 31/14; C12N 2770/20034

(57) ABSTRACT

The invention relates to a method and to a medicament for use in the treatment of COVID-19 and related pathologies, the medicament comprising or interacting with one or more conserved regions of at least 4 consecutive amino acids with a 100% match present in both the SARS-CoV-2 proteome and the human proteome, wherein the one or more conserved regions are preferably selected by: a. identification of one or more conserved regions of at least 4 consecutive amino acids with a 100% match between the SARS-CoV-2 proteome and the human proteome; b. identification of at least one pathway class from a systematic database comprising a plurality of human physiological pathway classes, each class comprising a plurality of human proteins that are functionally related to the said physiological pathway, wherein the said at least one pathway class shares at least one pathology and/or complication of the COVID-19 infection as a result of dysfunction in the said pathway and comprises at least one human protein comprising one or more conserved regions of at least 4 consecutive amino acids that have a 100% match with the SARS-CoV-2 proteome; and c. selecting the said identified one or more conserved regions for the preparation of the medicament. Based on this approach peptides were selected for the treatment of COVID-19 and several pathologies and complications that can occur in the context of COVID-19, but also as a separate disease, pathology, or complication. The invention also relates to a vaccine comprising one or more agents interacting with at least one region of at least 4 consecutive amino acids present in the SARS-CoV-2 proteome, not conserved between the SARS-CoV-2 proteome and the human proteome. Finally, the invention can provide a molecular and cellular explanation for the deviant infectivity, clinical behaviour, and pathology of emerging SARS-CoV-2 variants over time, to design vaccines to specifically prevent these complications, and to select peptides as therapeutic modality to treat these clinical manifestations and complications.

The invention relates to a medicament or pharmaceutical composition for treatment of COVID-19 and related pathologies.

Humans are suffering from outbreaks of Severe acute respiratory syndrome coronavirus (SARS-CoV) and Middle East respiratory syndrome coronavirus (MERS-CoV). A present pandemic viral outbreak is caused by Severe acute respiratory syndrome coronavirus-2 (SARS-CoV-2), which is a newly identified virus that differs from SARS-CoV and MERS-CoV but can cause largely similar symptomology associated with pneumonia. This viral disease was named “COVID-19” by the World Health Organization (WHO). The virus has spread to more than 200 countries and territories. WHO declared this disease to be a public health emergency of international concern. So far, there is no effective treatment for MERS-CoV, SARS-CoV or COVID-19.

Viral infection involves a large number of genomic and protein interactions, including protein-protein interactions between the virus and its host. These interactions range from the initial binding of viral coat proteins to host membrane receptors to the hijacking of the host transcription machinery by viral proteins.

In current methods to identify and prepare a medicine against a microbial pathogen, great efforts are made to understand the life cycle of the pathogen and the mode of interaction with the host. Modern medicine focuses on disturbing the interaction mechanism of the pathogen, or to provide an immunological response to the pathogen by via epitopes that can be recognized by the host e.g. with the aim to provide a vaccine.

The present inventors have now found a completely novel and surprising approach as to identify new medicaments or pharmaceutical compositions for treatment of COVID-19 and related pathologies. The new medicaments or pharmaceutical compositions could be developed by the contemplation that important and decisive peptide sequences of the pathogen playing a role in infectious diseases, are also present in proteins that play an important role in one or more physiological pathways in the host. The rationale behind this is that the pathogen, in order to be specific for a particular host, would preferably use proteins or maximum parts of proteins that are very similar to those of the host in order to interact with one or more biological pathways in the host. Such virus-host interaction may result in a particular pathology, clinical manifestations or complications in the host or even a variety of pathologies and complications. Herein, the terms ‘medicaments’ and ‘pharmaceutical composition’ can be used interchangeably.

In COVID-19 more and more pathologies and extrapulmonary clinical manifestations are reported over time, such as at brain and nervous system level (encephalitis, headache, ageusia, anosmia, encephalopathy, Guillain-Barre, stroke, myalgia, and other neurological problems e.g., memory problems, focusing problems, sleeping problems, gloom, fear, confusion, olfactory dysfunction, psychosis and other psychiatric problems, signs of Alzheimer disease and Parkinson's disease), at kidney level (acute kidney injury, proteinuria, haematuria, metabolic acidosis, electrolyte imbalance), at liver level (elevated aminotransferases, elevated conjugated bilirubin, low serum albumin), at lungs level (acute respiratory distress syndrome (ARDS), coagulopathy, pneumonia of varying severity), at gastrointestinal tract level (diarrhoea, nausea, vomiting, abdominal pain, mesenteric ischemia), at heart and cardiovascular level (myocardial injury, myocarditis, thromboembolism, endotheliitis, cardiac arrhythmias, cardiogenic shock, myocardial ischaemia, acute coronary syndrome, cardiomyopathy), at endocrine system level (hyperglycaemia, diabetic ketoacidosis), at skin level (petechiae, pernio-like skin lesions, livedo reticularis, erythematous rash, urticaria, vesicles), ischaemia-reperfusion problems and many others (Osuchowski et al.). The severe inflammatory response after ischaemia-reperfusion injury may also result in the systemic inflammatory response syndrome (SIRS) or the multiple organ dysfunction syndrome (MODS), which can account for up to 30-40% of intensive care unit mortality.

By their surprising approach, the inventors were capable to identify, by comparison of proteome data and physiological pathway analysis, a plurality of amino acid regions of at least 4 consecutive amino acids that can be used in the treatment of the infectious disease, in case of SARS-CoV-2 infection COVID-19, or for treatment of related pathologies. In this context the term ‘related pathologies’ refer to diseases that share one or more pathologies, clinical manifestations, complications or symptoms with those as brought about by the respective infection. The related diseases are not by infection by SARS-CoV-2, but are associated with similar pathologies, clinical manifestations, complications, or symptoms as those of a patient suffering from COVID-19. These pathologies, clinical manifestations, complications or symptoms can also relate to those of a different viral infection.

The inventors were for the first time capable of identifying, on the one hand, important peptide sequences (4-7 amino acids) of a pathogen that can be used to prepare a medicament against the said pathogen, whereas the said identification also enables the identification of endogenous pathogenic or host proteins sharing such peptide sequences, which proteins play important roles in one or more physiological pathways that may be disturbed when a host is suffering from a particular systemic disease. Such conserved peptide sequences or interaction therewith may therefore be useful in curing such a disease, in casu COVID-19. Conserved in this context means occurring in virtually all human beings, but also in microorganisms, in this case SARS-CoV-2.

The invention provides a method and a medicament or a pharmaceutical composition for use in a method for treatment of COVID-19 and related pathologies, the said medicament comprising or interacting with at least 4 consecutive amino acids present with a 100% match in both the SARS-CoV-2 proteome and the human proteome. The medicament preferably comprises one or a plurality of peptides each comprising one or more of the conserved regions described or defined herein. The peptides can have additional amino acid sequences and/or other chemical moieties, e.g. for carrier or targeting purposes. However, the peptide length is preferably 20 amino acids or less, more preferably 15 amino ids or less, even more preferably 10 amino acids or less, and most preferably the peptide consists of the conserved amino acid region.

The skilled person will be well capable of obtaining proteome information of the SARS-CoV-2 virus and man. To this end, numerous databases and sequence information as well as useful software tools are available by which regions of at least 4 consecutive amino acids can be identified that are shared among one or more proteins present in human and one or more proteins encoded by the viral genome. The term ‘conserved’ for the amino acid region herein means that the amino acids region has a 100% identity, i.e. a 100% match between the pathogen and the host.

In an attractive embodiment, the one or more conserved regions of at least 4 consecutive amino acids with a 100% match are selected by:
a. identification of one or more conserved regions of at least 4 consecutive amino acids with a 100% match between the SARS-CoV-2 proteome and the human proteome;
b. identification of at least one pathway class from a systematic database comprising a plurality of human physiological pathway classes (maps), each class comprising a plurality of human proteins that are functionally related to the said physiological pathway, wherein the said at least one pathway class shares at least one pathology and/or clinical manifestation and/or complication of the COVID-19 infection as a result of dysfunction in the said pathway and comprises at least one human protein comprising one or more conserved regions of at least 4 consecutive amino acids that have a 100% match with the SARS-CoV-2 proteome; and
c. selecting the said identified one or more conserved regions of at least 4 consecutive amino acids of step a., present in the at least one protein comprised in the pathway class identified in step b. for the preparation of the medicament.

In step a., conserved regions of at least 4 consecutive amino acids having a 100% match in both the human and the SARS-CoV-2 proteome are identified. Proteome data from both SARS-CoV-2 and humans are known. Thereto, the proteome data from both the host and the pathogen are compared in order to identify the conserved regions. The skilled person will be aware of suitable tools, that can e.g. be based on nucleic acid or amino acid sequence data.

Table 1 shows the distribution of shared conserved peptide sequences of the SARS-CoV-2 proteome and the human proteome, and the occurrence in the various SARS-CoV-2 open reading frames (ORFs). These data are based on the SARS-CoV-2 proteomes found in many different countries (number indicated) obtained from NCBI database (April 2020).

TABLE 1
Occurrence of conserved regions of 4-7 amino
acids (AA) in the SARS-CoV-2 proteome

| Peptide | distinct number | number of | from number |
|---|---|---|---|
| sequence | of shared | distinct | of distinct |
| length | sequences | ORF/proteins | countries |
| 4 | 2813 | 12 | 38 |
| 5 | 1146 | 12 | 38 |
| 6 | 136 | 11 | 38 |
| 7 | 11 | 5 | 33 |

Table 2 shows the length distribution of shared peptide sequences of the SARS-CoV-2 proteome and the human proteome and their occurrence in the various SARS-CoV2 proteins/ORFs (NCBI April 2020 database).

TABLE 2
Length distribution of shared peptide sequences
of the SARS-CoV-2 and human proteome

| Peptide | | | | | | |
|---|---|---|---|---|---|---|
| Length | ORF1a | ORF1ab | S protein | ORF3a | E protein | M protein |
| 4 | 1228 | 1939 | 392 | 149 | 24 | 90 |
| 5-7 | 539 | 801 | 158 | 90 | 13 | 23 |
| 6-7 | 51 | 69 | 14 | 20 | 2 | 1 |
| 7 | 2 | 3 | 0 | 2 | 0 | 0 |
| Peptide | | | | | | |
| Length | ORF6 | ORF7a | ORF7b | ORF8 | N protein | ORF10 |
| 4 | 39 | 52 | 29 | 43 | 169 | 28 |
| 5-7 | 18 | 19 | 14 | 19 | 134 | 10 |
| 6-7 | 1 | 2 | 3 | 2 | 33 | 0 |
| 7 | 0 | 0 | 0 | 0 | 6 | 0 |

Table 3 shows the number of shared SARS-CoV-2 and human conserved peptide sequences (length>4 AA) in the various SARS-CoV-2 proteins/ORFs. Data is obtained from NCBI database in April 2020 and in April 2021 of various countries. An increase in the numbers of shared peptides sequences is observed over time.

TABLE 3
The number of shared SARS-CoV-2 and humans conserved peptide
sequences (length > 4 AA) in the various SARS-CoV-2 proteins
present in the NCBI databases of April 2020 and April 2021

| | # Peptide | # Peptide | |
|---|---|---|---|
| SARS-COV-2 | Sequences | Sequences | |
| Protein Name | (April 2020) | (April 2021) | % Increase |
| ORF1a | 539 | 718 | 33% |
| ORF1ab | 801 | 1035 | 29% |
| S protein | 158 | 213 | 35% |
| ORF3a | 90 | 125 | 39% |
| E protein | 13 | 14 | 8% |
| M protein | 23 | 25 | 9% |
| ORF6 | 18 | 22 | 22% |
| ORF7a | 19 | 33 | 74% |
| ORF7b | 14 | 26 | 86% |
| ORF8 | 19 | 43 | 126% |
| N protein | 134 | 164 | 22% |
| ORF10 | 10 | 19 | 90% |
| Total peptide | 1838 | 2437 | 33% |
| sequences | | | |
| Total # Unique | 1293 | 1704 | 32% |
| Peptide | | | |
| Sequences | | | |

Table 4 shows conserved human peptide sequences that share a region of at least 5 consecutive amino acids with open reading frames of SARS-CoV-2.

TABLE 4
Human sequences with at least a 5 AA
overlapping sequence with the SARS-COV-2
proteome as of April 2020

| SARS- | SARS- | |
|---|---|---|
| COV-2 | COV-2 SEQ | Human peptide SEQ |
| Nucleo- | LLPAADLDD | AADLD, PAADL, LPAAD, AADLDD, |
| capsid | | LLPAADL |
| phospho- | SS(C/R)SS | SSCSS, SSRSR, SRSSS, SSSRSR, |
| protein | SRSR | CSSSRS, CSSSRSR, SSCSSSR, |
| | | SSRSSS |
| Surface | LVLLPLV | LLPLV, LVLLPL, VLLPL, VLLPLV |
| glyco- | GAGAAL | GAGAA, AGAAL |
| protein | VLPPLL | LPPLL, VLPPL, VLPPLL |
| ORF1ab | GGVAGA | GGVAG, GVAGA, GGVAG, GGVAGA |
| | AALGVL | ALGVL, AALGV, AALGVL |
| | DPAQLP | DPAQL, DPAQLP |
| | LLGVGG | LGVGG, LLGVG |
| | LGVLVP | LGVLV |
| | VLQAVGA | VLQAV, VLQAVG, LQAVG |
| | GVLPQLEQ | LPQLE, VLPQL, GVLPQ, PQLEQ |
| | VLQLPQG | QLPQG, LQLPQ, VLQLPQG |
| | ALPQRH | ALPQR, LPQRH |
| | ALQLEEE | LQLEE, ALQLE, ALQLEEE |
| | LPLQLG | LPLQL, LPLQLG |
| | WVADAV | VVADA, VVADAV |
| | RLALGG | LALGG, RLALGG |
| | LGGLHL | GGLHL, LGGLH, LGGLHL |
| ORF3a | ADGLEAP | ADGLE, DGLEA, GLEAP, ADGLEA |
| | QPEEEQE | EEEQE, PEEEQE, PEEEQ, PEEEQE, |
| | | QPEEEQ, QPEEEQ |
| | LLLVA(A/V) | AVGLE, LLVAV, LVAVG, VAVGL, |
| | (G/V)LE | VAVGLE, LLVAVG, LLVAAG, LLVAV, |
| | | LLLVA, LVAAV, LVAAG |
| | VL(L/D)LLV | LLVAA, LLLVA, LDLLV, LLVAV, |
| | A(A/V) | VLDLL, LLLLV, LLLVAA |

In step b, a pathway class is identified from a systemic database that comprises a plurality of human physiological pathways. The term ‘physiological pathway’, herein also ‘pathway’ is well known in the art, and is defined herein as a network of chemical reactions or interactions by a group of proteins in a cell that works together to control one or more cell function(s). Genes, gene products, or proteins are also classified based on their putative, predicted, or experimentally confirmed role in one or more particular biological function(s) such as cellular component (the locations relative to cellular structures in which a gene product or protein performs a function, either cellular compartments, or stable macromolecular complexes of which they are parts), molecular function (molecular-level activities performed by gene product or protein) or biological process (the larger processes, or ‘biological programs’ accomplished by multiple molecular activities) (Ashburner et al. Gene ontology: tool for the unification of biology. Nat Genet. May 2000; 25(1):25-9).

There are many databases wherein pathways can be classified. For example, the KEGG database (Kyoto Encyclopedia of Genes and Genomes: https://www.kegg.jp/), Reactome database, and the STRING database. Pathway classification databases can differ from one another in architecture and in definition of the classifications. All pathway classification databases are based on the above mentioned pathway definition, however, the distribution or grouping or classification of pathways to cellular- and biological processes, and biological systems, can be different between databases. Each of these databases discriminate a number of so-called ‘Top-levels’ from ‘Lower levels’. For example the Reactome database (Jassal B et al. Nucleic Acids Res. 2020 Jan. 8; 48(D1):D498-D503. doi: 10.1093/nar/gkz1031. PubMed PMID: 31691815) literature defines 27 top-levels (representing a broad area of biological- and cellular processes) covering one or more levels with group of pathways, becoming more specific with each lower level and ending up at lowest level presenting single pathway. These 27 top levels discriminated in mammalian molecular biology are: gene expression (transcription), cellular responses to external stimuli, DNA replication, disease, vesicle-mediated transport, extracellular matrix organization, haemostasis, transport of small molecules, chromatin organization, metabolism of proteins, developmental of biology, immune system, digestion and absorption, cell-cell communication, protein localization, programmed cell death, muscle contraction, reproduction, signal transduction, metabolism, neuronal system, autophagy, circadian clock, cell cycle, DNA repair, metabolism of RNA, and organelle biogenesis and maintenance. Another example of database is the KEGG PATHWAY database. The KEGG PATHWAY database is the central database in the category Systems Information, consisting of manually drawn KEGG pathway maps, hierarchically classified, representing knowledge of the molecular interaction, reaction, and relation networks for six biological systems, namely, 1. metabolism, 2. genetic information processing, 3. environmental information processing, 4. cellular processes, 5. organismal systems, and 6. human diseases (Top KEGG level 1). Each biological system contains a collection of pathway maps, representing wiring diagrams of proteins and other gene products responsible for various cellular functions involved in a biological system (KEGG level 2). In other words, KEGG level 2 pathway map is a molecular interaction/reaction network diagram of multiple KEGG pathways. The third level (KEGG level 3) in the hierarchy corresponds to individual pathway map and the fourth level (KEGG level 4) corresponds to genes/protein entries involved in an individual pathway. Accordingly, conserved peptide sequences selected from the SARS-CoV-2 proteome can be related to any of the pathways, as defined in the respective databases. Preferably, the envisaged one or more pathways are top-level pathways. In the SARS-CoV-2 proteome, some sequences can be identified that are most related to several ‘top-level’ pathways, while some other sequences are more specifically related to only some top-level pathways or a single top-level pathway. Based on the pathway or pathways whereto the said one or more proteins that comprise a conserved region of at least 4 consecutive amino acids belong, a medicament can be designed to cure a disease, and/or pathology, and/or clinical manifestation, and/or complication that is associated with dysfunction of the said pathway. Such a disease is herein also defined as a pathway-associated disease. When the conserved SARS-CoV-2 peptide sequence is present in one or more human proteins present in a large number of different pathway classes, in particular top-level pathway classes, such a conserved region is a good candidate as active ingredient or part of an active ingredient against COVID-19.

Starting with step a., a comparison is made between the proteomic sequences of the pathogen SARS-CoV-2 that are represented in the complete genome of the host. i.e. human. As a single continuous conserved peptide sequence is about 5 to 8 amino acids in length, peptide sequences of 5 to 7 amino acids can advantageously be analysed. The protein-protein interactions and pathway analyses of the KEGG database involving these conserved peptide sequences identified 118 pathways (KEGG level 3), all with a small false discovery rate (FOR) of ≤0.05. Pathways limited to those with an FOR cut-off of 10-4 are presented in Table 5

TABLE 5
Pathways selected from the whole human proteome on the basis of
SARS-CoV-2 shared conserved peptide sequences listed in Table 7

| | | observed | background | false |
|---|---|---|---|---|
| | KEGG class | gene | gene | discovery |
| KEGG Level 3 | PATH: | count | count | rate |
| Insulin signalling pathway | ko04910 | 18 | 134 | 2.13E−21 |
| Longevity regulating pathway | ko04211 | 15 | 88 | 2.98E−19 |
| Insulin resistance | ko04931 | 15 | 107 | 2.86E−18 |
| Glutamatergic synapse | ko04724 | 15 | 112 | 4.02E−18 |
| Platelet activation | ko04611 | 15 | 123 | 1.17E−17 |
| Apelin signalling pathway | ko04371 | 15 | 133 | 2.87E−17 |
| Oxytocin signally pathway | ko04921 | 15 | 149 | 1.19E−16 |
| Circadian entrainment | ko04713 | 13 | 93 | 4.14E−16 |
| Calcium signalling pathway | ko04020 | 15 | 179 | 1.19E−15 |
| Taste transduction | ko04742 | 12 | 81 | 3.30E−15 |
| Gap junction | ko04540 | 12 | 87 | 6.55E−15 |
| Dilated cardiomyopathy (DCM) | ko05414 | 12 | 88 | 6.81E−15 |
| Glucagon signalling pathway | ko04922 | 11 | 100 | 9.24E−13 |
| Adrenergic signally in cardiomyocytes | ko04261 | 11 | 139 | 2.46E−11 |
| Retrograde endocannabinoid signally | ko04723 | 11 | 148 | 4.37E−11 |
| Cholinergic synapse | ko04725 | 10 | 111 | 7.04E−11 |
| cGMP-PKG signally pathway | ko04022 | 11 | 160 | 8.60E−11 |
| AMPK signalling pathway | ko04152 | 10 | 120 | 1.30E−10 |
| Hypertrophic cardiomyopathy (HCM) | ko05410 | 9 | 81 | 1.30E−10 |
| Regulation of lipolysis in adipocytes | ko04923 | 8 | 53 | 1.85E−10 |
| Oestrogen signalling pathway | ko04915 | 10 | 133 | 2.90E−10 |
| Aldosterone synthesis and secretion | ko04925 | 9 | 93 | 3.45E−10 |
| Long-term depression | ko04730 | 8 | 60 | 4.00E−10 |
| cAMP signalling pathway | ko04024 | 11 | 195 | 4.65E−10 |
| Phospholipase D signalling pathway | ko04072 | 10 | 145 | 5.47E−10 |
| Serotonergic synapse | ko04072 | 9 | 112 | 1.38E−09 |
| Thermogenesis | ko04714 | 11 | 228 | 2.03E−09 |
| Vascular smooth muscle contraction | ko04270 | 9 | 119 | 2.13E−09 |
| Autophagy - animal | | 9 | 125 | 3.10E−09 |
| Salivary secretion | ko04970 | 8 | 86 | 4.36E−09 |
| Endocrine resistance | | 8 | 95 | 8.81E−09 |
| Cortisol synthesis and secretion | hsa04927 | 7 | 63 | 1.58E−08 |
| Long-term potentiation | ko04720 | 7 | 64 | 1.70E−08 |
| Type II diabetes mellitus | hsa04930 | 6 | 46 | 8.81E−08 |
| Proteoglycans in cancer | hsa05205 | 9 | 195 | 1.04E−07 |
| Morphine addiction | hsa05032 | 7 | 91 | 1.51E−07 |
| Inflammatory mediator regulation | hsa04750 | 7 | 92 | 1.58E−07 |
| of TRP channels | | | | |
| Pancreatic secretion | hsa04972 | 7 | 95 | 1.90E−07 |
| Renin secretion | hsa04924 | 6 | 63 | 4.34E−07 |
| Adipocytokine signalling pathway | hsa04920 | 6 | 69 | 7.01E−07 |
| Gastric acid secretion | hsa04971 | 6 | 72 | 8.66E−07 |
| Arrhythmogenic right ventricular | | 6 | 72 | 8.66E−07 |
| cardiomyopathy | | | | |
| Thyroid hormone synthesis | | 6 | 73 | 8.92E−07 |
| Cardiac muscle contraction | | 6 | 76 | 1.09E−06 |
| Dopaminergic synapse | | 7 | 128 | 1.11E−06 |
| EGFR tyrosine kinase inhibitor | | 6 | 78 | 1.20E−06 |
| resistance | | | | |
| Relaxin signalling pathway | | 7 | 130 | 1.20E−06 |
| Rap1 signalling pathway | | 8 | 203 | 1.56E−06 |
| Insulin secretion | | 6 | 84 | 1.71E−06 |
| MAPK signalling pathway | | 9 | 293 | 2.07E−06 |
| GABAergic synapse | | 6 | 88 | 2.13E−06 |
| GnRH signalling pathway | | 6 | 88 | 2.13E−06 |
| Endocrine/other factor-regulated | | 5 | 47 | 2.38E−06 |
| calcium reabsorption | | | | |
| Ovarian steroidogenesis | | 5 | 49 | 2.83E−06 |
| Cushing's syndrome | | 7 | 153 | 2.87E−06 |
| Chemokine signalling pathway | | 7 | 181 | 8.28E−06 |
| Thyroid hormone signalling pathway | | 6 | 115 | 8.40E−06 |
| Oocyte meiosis | | 6 | 116 | 8.66E−06 |
| Starch and sucrose metabolism | | 4 | 33 | 1.82E−05 |
| Pathways in cancer | | 10 | 515 | 2.21E−05 |
| Human papillomavirus infection | | 8 | 317 | 3.04E−05 |
| Progesterone-mediated oocyte maturation | | 5 | 94 | 4.98E−05 |
| PI3K-Akt signalling pathway | | 8 | 348 | 5.68E−05 |
| Tight junction | | 6 | 167 | 5.82E−05 |
| Melanogenesis | | 5 | 98 | 5.82E−05 |
| Neuroactive ligand-receptor interaction | | 7 | 272 | 9.16E−05 |

Analysis of the first 40 pathways from Table 5 (with an FOR ranging from 2.13×10⁻²¹ to 8.66×10⁻⁷) and the pathways that according to the KEGG database are related to these first 40 pathways shows that their effects are not only associated with the clinical manifestations and/or complications of COVID-19, but also are directly or indirectly related to the underlying cellular processes and their functional manifestations, as shown in Table 6.

TABLE 6
Pathway pathologies, related pathways, cellular processes and manifestations

| Pathology/ | Related pathways, underlying cellular |
|---|---|
| complications | processes and functional manifestations |
| Neurological | Glutamatergic synapse, Cholinergic synapse, Long-term |
| | depression, Longevity regulating pathway, Long-term potentiation, |
| | Dopaminergic synapse, GABAergic synapse, GnRH signalling |
| | pathway, Alzheimer's disease. |
| Gustatory | Taste transduction, Salivary secretion |
| Renal | Cortisol synthesis and secretion, Renin secretion, AMPK signalling |
| | pathway, mTOR signalling pathway, Circadian entrainment, |
| | Vasopressin-regulated water reabsorption, Longevity regulating |
| | pathway |
| Temperature | Thermogenesis pathway |
| Inflammation | Insulin signalling pathway, Longevity regulating pathway, Platelet |
| | activation, cGMP-PKG signalling pathway, Metabolic pathways, |
| | Oestrogen signalling pathway, Phospholipase D signalling pathway, |
| | Cortisol synthesis and secretion, AMPK signalling pathway, mTOR |
| | signalling pathway, Inflammatory mediator regulation of TRP |
| | channels, Rap1 signalling pathway, MAPK signalling pathway, |
| | Chemokine signalling pathway, PI3K-Akt signalling pathway, |
| | Sphingolipid signalling pathway, Fc gamma R-mediated |
| | phagocytosis, Natural killer cell mediated cytotoxicity, Necroptosis, |
| | Fc epsilon RI signalling pathway, Viral myocarditis, Autophagy - |
| | animal, Circadian entrainment |
| Cardiovascular | Dilated cardiomyopathy (DCM), Insulin signalling pathway, Apelin |
| | signalling pathway, Longevity regulating pathway, Vascular smooth |
| | muscle contraction, Renin secretion, Arrhythmogenic right |
| | ventricular cardiomyopathy, Cardiac muscle contraction, Relaxin |
| | signalling pathway, Insulin secretion, mTOR signalling pathway, |
| | AMPK signalling pathway, Vasopressin-regulated water |
| | reabsorption, Metabolic pathways |
| BMI (obesity) | Insulin resistance, Insulin signalling pathway, Glucagon signalling |
| | pathway, AMPK signalling pathway, Endocrine resistance, |
| | Pancreatic secretion, Thermogenesis pathway, PI3K-Akt signalling |
| | pathway, Adipocytokine signalling pathway, Gastric acid secretion, |
| | Insulin secretion, Ovarian steroidogenesis, Circadian entrainment, |
| | Type II diabetes mellitus, Carbohydrate digestion and absorption, |
| | Metabolic pathways |
| Psychiatry | Oxytocin signalling pathway, Circadian entrainment, Longevity |
| | regulating pathway, Serotonergic synapse, Dopaminergic synapse, |
| | Glutamatergic synapse, GABAergic synapse, GnRH signalling |
| | pathway, Long-term potentiation, Long-term depression |
| Addiction and pain | Morphine addiction, Amphetamine addiction, Cocaine addiction, |
| | Alcoholism, Nicotine addiction, Glutamatergic synapse, |
| | Serotonergic synapse, Dopaminergic synapse, GABAergic synapse |

It was found that SARS-CoV-2 shared conserved peptide sequences in human proteins occur in several pathways, and other conserved peptide sequences in fewer pathways or even in a single pathway. The peptides identified in this manner are listed in Table 7 in the column Shared SEQ. The values in the other columns represent the proportion of pathways involved in that (patho)physiological- or biological process that have one or more proteins with at least one shared peptide. The value zero indicates that that particular peptide does not show up in this analysis.

TABLE 7
SARS-COV-2-shared conserved peptide
sequences (SEQ) in human pathways
involved in four (patho)physio-
logical or biological process

| | | | Viral | Viral |
|---|---|---|---|---|
| Shared | COVID- | Longevity- | life | inte- |
| SEQ | 19 | aging | cycle | gration |
| AADLD | 29 | 0 | 0 | 0 |
| AADLDD | 26 | 0 | 0 | 0 |
| AADPA | 0 | 48 | 37 | 0 |
| AALGV | 100 | 32 | 0 | 0 |
| AALGVL | 100 | 0 | 0 | 91 |
| ADLDD | 26 | 0 | 0 | 0 |
| AGAAL | 26 | 0 | 0 | 0 |
| ALGVL | 100 | 69 | 0 | 91 |
| ALLLL | 6 | 30 | 79 | 91 |
| AVGLE | 68 | 32 | 26 | 55 |
| AVLLA | 0 | 42 | 74 | 0 |
| CSSSRS | 3 | 0 | 0 | 64 |
| DGVDV | 0 | 38 | 74 | 0 |
| EEAAR | 0 | 18 | 32 | 0 |
| EEEQE | 12 | 28 | 0 | 0 |
| EQEED | 0 | 0 | 26 | 0 |
| FIKRS | 0 | 90 | 0 | 0 |
| FSSIF | 0 | 0 | 89 | 0 |
| GAGAA | 26 | 32 | 42 | 0 |
| GAGAAL | 26 | 0 | 0 | 0 |
| GDAAL | 0 | 0 | 26 | 0 |
| GGLHL | 26 | 0 | 0 | 0 |
| GGLHLL | 26 | 0 | 0 | 64 |
| GLAAV | 0 | 46 | 47 | 0 |
| GLHLL | 26 | 10 | 0 | 64 |
| GVLPQ | 76 | 0 | 0 | 0 |
| GVLPQL | 76 | 0 | 0 | 0 |
| HLLLVA | 9 | 0 | 0 | 55 |
| HLLLVAA | 9 | 0 | 0 | 55 |
| IIFWF | 0 | 0 | 0 | 27 |
| IIFWFS | 0 | 0 | 0 | 27 |
| KIKTI | 0 | 46 | 0 | 0 |
| LALGG | 21 | 11 | 0 | 27 |
| LALLL | 6 | 0 | 0 | 91 |
| LALLLL | 6 | 0 | 0 | 91 |
| LDLLV | 38 | 11 | 0 | 0 |
| LDRLE | 0 | 34 | 0 | 0 |
| LGGLH | 24 | 0 | 5 | 64 |
| LGGLHL | 24 | 0 | 0 | 64 |
| LGLLP | 0 | 27 | 0 | 0 |
| LGVLV | 26 | 10 | 16 | 0 |
| LGVLVP | 26 | 0 | 0 | 0 |
| LHVVG | 0 | 0 | 37 | 0 |
| LLLLV | 0 | 56 | 100 | 0 |
| LLLQL | 0 | 6 | 32 | 0 |
| LLLVA | 74 | 17 | 32 | 100 |
| LLLVAA | 74 | 0 | 0 | 100 |
| LLPLV | 24 | 0 | 16 | 91 |
| LLQAP | 12 | 7 | 68 | 0 |
| LLVAA | 85 | 4 | 16 | 0 |
| LLVAAG | 26 | 0 | 16 | 0 |
| LPLQL | 26 | 0 | 0 | 0 |
| LPLQLG | 26 | 0 | 0 | 0 |
| LPVLQV | 12 | 4 | 0 | 27 |
| LQAVG | 9 | 0 | 26 | 0 |
| LQLPQ | 68 | 0 | 0 | 0 |
| LTSFS | 0 | 0 | 37 | 0 |
| LVAVG | 0 | 14 | 32 | 0 |
| LVGVL | 0 | 8 | 42 | 0 |
| LVLLL | 0 | 10 | 26 | 0 |
| LVLLP | 3 | 18 | 32 | 64 |
| LVLLPL | 3 | 0 | 5 | 64 |
| PAADL | 29 | 0 | 0 | 0 |
| PAADLD | 29 | 0 | 0 | 0 |
| PLVEQ | 0 | 0 | 26 | 0 |
| PVPEV | 0 | 56 | 0 | 0 |
| QLPQG | 68 | 31 | 0 | 36 |
| QPGVA | 0 | 0 | 26 | 0 |
| RLALGG | 21 | 0 | 0 | 27 |
| RQVLL | 0 | 46 | 0 | 0 |
| SCSSS | 32 | 0 | 0 | 73 |
| SCSSSR | 32 | 0 | 0 | 73 |
| SSCSS | 32 | 0 | 21 | 0 |
| SSCSSS | 32 | 0 | 0 | 73 |
| SSCSSSR | 32 | 0 | 0 | 73 |
| SSLSS | 0 | 35 | 32 | 0 |
| SSRSR | 3 | 0 | 0 | 64 |
| SSRSSS | 24 | 0 | 0 | 64 |
| SSSRS | 3 | 13 | 0 | 64 |
| SSSRSR | 3 | 0 | 0 | 64 |
| TFSSI | 0 | 0 | 89 | 0 |
| TFSSIF | 0 | 0 | 89 | 0 |
| VAAVL | 0 | 24 | 26 | 0 |
| VADGL | 0 | 100 | 79 | 0 |
| VAVGL | 68 | 7 | 5 | 0 |
| VAVGLE | 68 | 0 | 0 | 55 |
| VDGQV | 0 | 0 | 26 | 0 |
| VGVAL | 0 | 10 | 26 | 0 |
| VLDLL | 38 | 14 | 0 | 0 |
| VLDLLV | 38 | 0 | 0 | 0 |
| VLGLA | 18 | 11 | 0 | 27 |
| VLGLAA | 18 | 0 | 0 | 27 |
| VLLLL | 0 | 18 | 26 | 0 |
| VLLPL | 26 | 8 | 5 | 0 |
| VLLPLV | 24 | 0 | 0 | 91 |
| VLPQL | 76 | 0 | 21 | 0 |
| VLQAV | 9 | 0 | 11 | 64 |
| VLQAVG | 9 | 0 | 0 | 64 |
| VLQLP | 68 | 1 | 0 | 0 |
| VLQLPQ | 68 | 0 | 0 | 0 |
| VLQLPQG | 68 | 0 | 0 | 36 |
| VLQPE | 0 | 32 | 32 | 0 |
| VLVPL | 0 | 93 | 0 | 0 |
| VVEVV | 0 | 34 | 21 | 0 |

The 5-, 6- and 7-mer peptide sequences were identified (NCBI April 2020 database) that include a 5-mer sequence only, shown in Table 7, were selected from proteins that are found to be directly or indirectly related with the pathways metabolism, longevity and biological aging as well as associated with known clinical manifestations and complications of COVID-19 (Tables 5 and 6).

A high significance was observed for the biological processes longevity and aging-related pathway classes, as well as for the biological process viral life cycle-related pathway class in COVID-19.

In an attractive embodiment of the invention, it is also possible to identify one or more biological processes of the host first, and then check for conserved regions of at least 4 amino acids present in both one or more proteins of the said biological class, and the genome of the pathogen. Accordingly, the above step a. of identification of one or more conserved regions between the SARS-CoV-2 proteome and the human proteome is replaced by the following steps a1 and a2:
step a1. comprising identification of one or more biological processes from a systematic database, the database comprising a plurality of human biological processes and, for each biological process, a plurality of human proteins that are functionally related to the said biological process, and
step a2. comprising identification of at least one conserved region of at least 4 consecutive amino acids in one or more of the plurality of proteins identified in step a1., said conserved region of at least 4 consecutive amino acids having a 100% match with the SARS-CoV-2 proteome.
   A biological process is defined as a series of molecular events, with a defined beginning and end; biological processes are independent and do not represent the interactions among molecules, whereas a biological pathway is a network of chemical reactions, and one or more thereof can be part of the biological process (Gene Ontology, Nat. Genet. May 2000; 25(1): 25-29). Databases comprising a plurality of biological processes and a plurality of proteins that are categorized among the different process classes according to the functionality of the said proteins are known in the art, such as e.g. the gene Ontology Database (supra).

Accordingly, an inventory of one or more significant or otherwise envisaged classes of biological processes can be made e.g. in order to identify one or more regions of at least 4 consecutive amino acids present in the said one or more classes of biological process, in order to be used as an active ingredient in a medicament against an envisaged disease. In that case, an inventory of amino acid regions of at least 4 consecutive amino acids present in proteins belonging to the envisaged one or more classes of biological processes is made, followed by a comparison with the SARS-CoV-2 proteome, and identification of one or more biological pathways as described above. Said pathways are preferably identified among pathways, known to be involved in the said biological process. This way, the identification of conserved amino acid sequences is preceded by a functional analysis instead of starting with a proteome comparison.

These above-mentioned biological processes of longevity and aging, viral life cycle, and viral integration were chosen for this approach, i.e. starting with these biological class. According to this approach, it is also possible to identify one or more subclasses, based on a first identification of a biological process, followed by identification of one or more conserved regions of at least 4, preferably at least 5 identical consecutive amino acids in proteins of the biological processes. Those conserved regions then serve as starting point for the identification of one or more pathway subclasses according to the above.

Starting from the biological process class of longevity and ageing, 274 human Swiss-Prot proteins from the Gene Ontology database, related to the biological processes term ‘aging’ were retrieved. 191 proteins thereof contain at least one 5- and/or 6-mer that also occurs in one or more SARS-CoV-2 proteins. 341 conserved peptide sequences with one or more 5- and/or 6-mers were shared by the 191 human proteins involved in the ‘aging’ and SARS-CoV-2 proteins. The protein-protein interactions and pathways analyses tools (e.g. String database, KEGG database) involving these peptides identified 129 pathways, all with an FDR smaller than 0.05. Table 8 shows these with an FDR of less than 10⁻⁴.

The results show that some SARS-CoV-2 shared conserved peptide sequences in human proteins occur in several pathways, and other peptide sequences in fewer pathways or even in a single pathway. The peptide sequences identified in this manner are listed in Table 7 in the column Longevity/aging.

Dysfunctions associated with the most significant sub-pathway of AGE-RAGE signalling-mediated diabetic complications include diabetic neuropathy, diabetic nephropathy, diabetic vascular complications, and diabetic foot syndrome. In addition, advanced glycation end products (AGEs) also play an important role in most age-related diseases such as Alzheimer's, cancer, cardiovascular disease, kidney disease, high blood pressure, stroke, visual impairment, and skin diseases. In many cases of COVID-19, not only after intensive care treatment, patients suffer for months of memory problems, focusing problems, sleeping problems, gloom, fear and muscle weakness, all symptoms associated with ageing. These late effects may well be related to SARS-CoV-2 affected pathways shown in Table 5, e.g. oxytocin signalling, circadian entrainment, cholinergic synapse, long-term depression, and serotonergic synapse.

TABLE 8
Human pathways selected on the basis of
5- and 6-mer SARS-Cov-2 sequences present
in human proteins related to biological
process aging and longevity based on
sequences of Table 7

| | | false |
|---|---|---|
| | count in | discovery |
| Pathways | gene set | rate |
| AGE-RAGE signalling pathway | 14 of 98 | 8.64E−10 |
| in diabetic complications | | |
| FoxO signalling pathway | 15 of 130 | 1.16E−09 |
| Cellular senescence | 14 of 156 | 8.25E−08 |
| Apoptosis | 13 of 135 | 1.13E−07 |
| Pathways in cancer | 23 of 515 | 1.13E−07 |
| Longevity regulating pathway | 11 of 88 | 1.14E−07 |
| HTLV-I infection | 16 of 250 | 2.16E−07 |
| Pancreatic cancer | 10 of 74 | 2.33E−07 |
| Colorectal cancer | 10 of 85 | 6.91E−07 |
| Acute myeloid leukaemia | 9 of 66 | 9.95E−07 |
| HIF-1 signalling pathway | 10 of 98 | 1.95E−06 |
| Relaxin signalling pathway | 11 of 130 | 2.35E−06 |
| MAPK signalling pathway | 15 of 293 | 5.59E−06 |
| Neurotrophin signalling | 10 of 116 | 6.56E−06 |
| pathway | | |
| Viral carcinogenesis | 12 of 183 | 6.62E−06 |
| MicroRNAs in cancer | 11 of 149 | 6.62E−06 |
| AMPK signalling pathway | 10 of 120 | 6.92E−06 |
| Central carbon metabolism in | 8 of 65 | 6.92E−06 |
| cancer | | |
| Prolactin signalling pathway | 8 of 69 | 9.38E−06 |
| Chagas disease (American | 9 of 101 | 1.38E−05 |
| trypanosomiasis) | | |
| Oestrogen signalling pathway | 10 of 133 | 1.41E−05 |
| Chronic myeloid leukaemia | 8 of 76 | 1.59E−05 |
| EGFR tyrosine kinase | 8 of 78 | 1.82E−05 |
| inhibitor resistance | | |
| Insulin resistance | 9 of 107 | 1.82E−05 |
| TNF signalling pathway | 9 of 108 | 1.85E−05 |
| Hepatitis B | 10 of 142 | 1.98E−05 |
| Kaposi's sarcoma-associated | 11 of 183 | 2.52E−05 |
| herpesvirus infection | | |
| Endometrial cancer | 7 of 58 | 2.52E−05 |
| Gastric cancer | 10 of 147 | 2.52E−05 |
| Longevity regulating | 7 of 61 | 3.19E−05 |
| pathway - multiple species | | |
| Human papillomavirus | 14 of 317 | 3.19E−05 |
| infection | | |
| Osteoclast differentiation | 9 of 124 | 4.19E−05 |
| CGMP-PKG signalling | 10 of 160 | 4.26E−05 |
| pathway | | |
| Non-small cell lung cancer | 7 of 66 | 4.54E−05 |
| Hepatocellular carcinoma | 10 of 163 | 4.70E−05 |
| Glioma | 7 of 68 | 5.14E−05 |
| Prostate cancer | 8 of 97 | 5.14E−05 |
| Adipocytokine signalling | 7 of 69 | 5.32E−05 |
| pathway | | |
| B cell receptor signalling | 7 of 71 | 6.17E−05 |
| pathway | | |
| Cocaine addiction | 6 of 49 | 8.55E−05 |
| Chemokine signalling | 10 of 181 | 9.54E−05 |
| pathway | | |

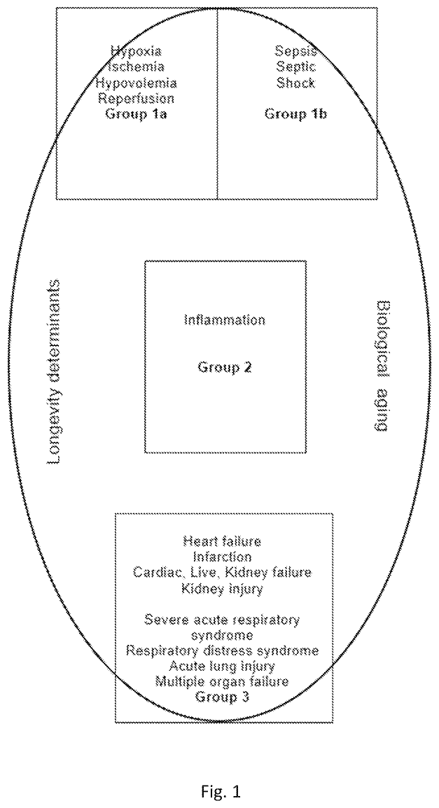

A simplified scheme of the sequence of (patho)physiological events leading to various pathological conditions of which the outcome is determined by the genes or their (by)products related to longevity pathways and aging is given in FIG. 1. Various endogenous and exogenous “insults”, such as either non-microbes (group 1a), e.g. hypoxic, ischemic, hypovolemic or reperfusion events, or as microbes (group 1b), e.g. bacteria or virus in sepsis and septic shock, lead to inflammatory responses (group 2). These responses can be beneficial or detrimental to various organs (group 3). The outcome of these responses is dependent on the various determinants of longevity pathways and biological aging.

Starting from the biological process class of viral life cycle, 210 human Swiss-Prot proteins were retrieved from Gene Ontology database, related to the biological processes term ‘viral life cycle’. Proteins related to the viral life cycle are proteins that influence a set of processes which all viruses follow to ensure their multiplication and survival. These include attachment and entry of the virus particle, decoding of genomic information, translation of viral mRNA by host ribosomes, genome replication, and assembly and release of viral particles containing the genome. 137 of the said 210 proteins contain at least two 5- and/or 6-mers that also occur in the various SARS-CoV-2 proteins, see Table 9.

TABLE 9
Number of distinct 5- and 6-mers conserved peptide sequences from
SARS-CoV-2 coded proteins that also occur at least twice in human
proteins related to ‘viral life cycle’ biological process

| Sars-CoV-2 | Number of |
|---|---|
| protein name | distinct peptides |
| S surface glycoprotein | 30 |
| E envelope protein | 6 |
| M membrane protein | 5 |
| N nucleocapsid | 32 |
| phosphoprotein | |
| ORF1a | 122 |
| ORF1ab | 174 |
| ORF3a | 68 |
| ORF6 | 12 |
| ORF7a | 1 |
| ORF7b | 4 |
| ORF8 | 7 |

The protein-protein interactions and pathways analyses tools (e.g. String database, KEGG database) involving these peptides identified 39 pathways, all with an FDR smaller than 0.05. Table 10 shows those having an FDR of less than 10⁻⁴. The top 2 pathways of this list concern endocytosis with an FDR of 2.40×10⁻²² and RNA transport with an FDR of 8.08×10⁻¹⁵. The results show that some SARS-CoV-2 shared peptide sequences in human proteins occur in several pathways, and other peptide sequences in fewer pathways or even in a single pathway. The peptides identified in this manner are listed in Table 7 in the column Viral life cycle. The values in this column represent the proportion of pathways that have one or more proteins with at least one shared peptide. The value zero indicates that that particular peptide does not show up in this analysis.

The human proteins containing conserved SARS-CoV-2 peptide sequences involved in the ‘viral life cycle’ appeared to be also involved in a number of other biological processes and diseases, see Table 10. These shared 5- and 6-mer peptide sequences can be synthesized according to known procedures and used not only as pharmaceuticals to modulate the biological processes involved in ‘viral life cycle’, but also to modulate several other biological processes and related diseases based on pathways mentioned in Table 10. A COVID-19 related pathology also encompasses other viral infections, that share one or more pathologies/complications.

TABLE 10
Human pathways shared with the pathways involved in
the virus life cycle selected on the basis of the
5- and 6-mer peptide sequences listed in Table 7

| | count in | false |
|---|---|---|
| KEGG pathway | gene set | discovery rate |
| Endocytosis | 27 of 242 | 2.40E−22 |
| RNA transport | 18 of 159 | 8.08E−15 |
| Cell adhesion molecules | 11 of 139 | 2.03E−07 |
| Phagosome | 11 of 145 | 2.30E−07 |
| Arrhythmogenic right | 8 of 72 | 1.52E−06 |
| ventricular cardiomyopathy | | |
| Hypertrophic cardiomyopathy | 8 of 81 | 2.94E−06 |
| ECM-receptor interaction | 8 of 81 | 2.94E−06 |
| Focal adhesion | 11 of 197 | 2.94E−06 |
| Necroptosis | 10 of 155 | 2.94E−06 |
| Dilated cardiomyopathy (DCM) | 8 of 88 | 3.18E−06 |
| Hematopoietic cell lineage | 8 of 94 | 4.63E−06 |
| Proteoglycans in cancer | 9 of 195 | 9.62E−05 |

Starting from the biological process class of viral integration, conserved 5-, 6- and 7-mer SARS-CoV-2 peptide sequences that include a 5-mer sequence only, that were related to the viral integration were identified. According to the Gene Ontology database, the related terms to viral integration are following: RNA polymerase, RNA polymerase I (involved in chain elongation, promoter clearance, promotor escape, transcription, transcription initiation and transcription termination), RNA polymerase II (involved in pre-transcription events, transcription pre-initiation and promotor opening, transcription, transcription initiation and promotor clearance, transcription elongation, transcription termination, transcribes snRNA genes, HIV promotor escape, promotor escape), RNA polymerase III (involved in transcription, transcription initiation, transcription initiation from type 1, type 2 and type 3 promotor, abortive and retractive initiation, chain elongation, termination), Influenza viral RNA transcription and replication, host interactions with influenza factors, influenza infection, influenza life cycle, influenza A, Epstein-Barr virus infection, integration of provirus, influenza viral RNA transcription and replication, antiviral mechanism by IFN-stimulated genes, export of viral ribonucleoproteins from nucleus, ISG15 antiviral mechanism, viral messenger RNA synthesis, viral carcinogenesis, viral myocarditis and viral mRNA translation. Based on the human proteins related to the ‘viral integration’ that contain SARS-CoV-2 peptide sequences 28 KEGG pathways were identified (Table 11).

The results show that some SARS-CoV-2 shared peptide sequences in human proteins occur in several pathways, and other peptide sequences in fewer pathways or even in a single pathway. The peptides identified in this manner are listed in Table 7 in the column Viral integration. The values in this column represent the proportion of pathways that have one or more proteins with at least one peptide shared with SARS-CoV-2. The value zero indicates that that particular peptide does not show up in this analysis.

TABLE 11
Human pathways selected on the basis of SARS-CoV-2 peptide
sequences related to the viral integration into the host
cell pathways based on the sequence listed in Table 7

| | count in | false |
|---|---|---|
| KEGG pathway | gene set | discovery rate |
| Epstein-Barr virus infection | 35 of 194 | 6.27E−08 |
| Herpes simplex infection | 25 of 181 | 0.00092 |
| Viral carcinogenesis | 24 of 183 | 0.002 |
| HIF-1 signalling pathway | 16 of 98 | 0.0033 |
| Influenza A | 22 of 168 | 0.0033 |
| Apelin signalling pathway | 18 of 133 | 0.0073 |
| Measles | 18 of 133 | 0.0073 |
| Human papillomavirus infection | 31 of 317 | 0.0086 |
| Toxoplasmosis | 15 of 109 | 0.0143 |
| Pathways in cancer | 42 of 515 | 0.0222 |
| Spliceosome | 16 of 130 | 0.023 |
| FoxO signalling pathway | 16 of 130 | 0.023 |
| RNA transport | 18 of 159 | 0.0232 |
| Insulin signalling pathway | 16 of 134 | 0.0242 |
| Platelet activation | 15 of 123 | 0.0262 |
| Tight junction | 18 of 167 | 0.0311 |
| EGFR tyrosine kinase inhibitor | 11 of 78 | 0.0342 |
| resistance | | |
| ABC transporters | 8 of 44 | 0.0342 |
| Hepatitis B | 16 of 142 | 0.0342 |
| Rap1 signalling pathway | 20 of 203 | 0.0375 |
| NF-kappa B signalling pathway | 12 of 93 | 0.0375 |
| Jak-STAT signalling pathway | 17 of 160 | 0.0375 |
| Endocrine resistance | 12 of 95 | 0.0382 |
| ErbB signalling pathway | 11 of 83 | 0.0385 |
| Adherens junction | 10 of 71 | 0.0385 |
| Osteoclast differentiation | 14 of 124 | 0.0407 |
| Ras signalling pathway | 21 of 228 | 0.0456 |
| Pancreatic cancer | 10 of 74 | 0.0456 |

Anti-viral peptide sequences identified herein can be synthesized according to known procedures and used for the anti-viral treatment of COVID-19 patients, and patients with other viral diseases. These pharmaceuticals will also be useful for the treatment of animals with a viral infection. Similar analyses can be done for any other biological process, molecular function and cellular component starting from the related human proteins listed in e.g. the Gene Ontology database.

In an attractive embodiment of the invention, it is also possible to identify one or more biological systems of the host first, and then check for conserved regions of at least 4 amino acids present in both on or more proteins of the said biological system, and in the genome of the pathogen. Accordingly, the above step a. of identification of one or more conserved regions between SARS-CoV-2 proteome and the human proteome is replaced by the following steps a3 and a4:
step a3. comprising identification of one or more biological system from a systematic database, the database comprising a plurality of human biological systems and, for each biological system, a plurality of human pathway maps representing wiring diagrams of proteins and other gene products responsible for various cellular functions involved in a biological system, and
step a4. comprising identification of at least one conserved region of at least 4 consecutive amino acids in one or more of the plurality of proteins identified in step a3., said conserved region of at least 4 consecutive amino acids having a 100% match with the SARS-CoV-2 proteome.

KEGG is a manually curated resource integrating eighteen databases categorized into systems, genomic, chemical and health information. The content covers wide-ranging biological objects, including molecular interaction/reaction/relation networks (systems information), genes and proteins (genomic information), chemical substances and reactions (chemical information), and human diseases and drugs (health information). The important aspect in the overall architecture of KEGG is network hierarchy.

The KEGG PATHWAY database is the central database in the category Systems Information, consisting of manually drawn KEGG pathway maps, hierarchically classified, representing knowledge of the molecular interaction, reaction, and relation networks for six biological systems, namely, 1. metabolism, 2. genetic information processing, 3. environmental information processing, 4. cellular processes, 5. organismal systems, and 6. human diseases (Top KEGG level 1). Each biological system contains a collection of pathway maps, representing wiring diagrams of proteins and other gene products responsible for various cellular functions involved in a biological system (KEGG level 2). In other words, KEGG level 2 pathway map is a molecular interaction/reaction network diagram of multiple KEGG pathways. The third level (KEGG level 3) in the hierarchy corresponds to individual pathway maps and the fourth level (KEGG level 4) corresponds to genes/protein entries involved in an individual pathway.

As shown in Table 3, in April 2020 1293 unique peptide sequences (length>4AA) were shared between SARS-CoV-2 and the human proteome. Of these 1293 peptide sequences, 845 peptide sequences can be linked to 1436 human genes (KEGG level 4) that are associated with the pathways (KEGG level 3) in KEGG database. These numbers have increased overtime due to mutations. As of April 2021, 1704 unique peptide sequences (length>4AA) were shared between SARS-CoV-2 and the human proteome. Of these 1704 peptide sequences, 1085 peptide sequences can be linked to some 1715 human genes (KEGG level 4) that are associated with around 300 pathways (KEGG level 3) in KEGG database. To get an overview of this huge data, the data were distributed as shown in Table 12, wherein the vertical columns 1-6 represent the biological systems 1. Metabolism, 2. Genetic information processing, 3. Environmental information processing, 4. Cellular processes, 5. Organismal systems and 6. Human diseases (Top KEGG level 1). Within the column, the shaded area shows the number of KEGG level 2 pathway maps that are identified for that biological system based on the conserved peptide sequences shared by the human and SARS-CoV-2 proteome. The other figures in the vertical columns represent the numbers of pathway maps that are shared with the other indicated biological systems (KEGG level 1).

TABLE 12
Identification of numbers of KGG level 2 pathways within the 6
top KEGG level 1 biological systems, based on the KEGG database

| KEGG Level 1 (FDR <= 0.001) | 1 | 2 | 3 | 4 | 5 | 6 |
|---|---|---|---|---|---|---|
| 1. Metabolism | 54 | 1 | 6 | 1 | 3 | 6 |
| 2. Genetic Information Processing | 0 | 18 | 1 | 1 | 0 | 1 |
| 3. Environmental Information Processing | 10 | 3 | 31 | 24 | 26 | 27 |
| 4. Cellular Processes | 2 | 3 | 14 | 18 | 11 | 16 |
| 5. Organismal Systems | 38 | 1 | 66 | 59 | 76 | 62 |
| 6. Human Diseases | 8 | 3 | 65 | 61 | 61 | 72 |

The skilled person will be able to retrieve additional information from the KEGG database, such as other pathways that are related to the primary pathways shown in Table 5. In Table 13 the analysis of biological system (KEGG level 1) is shown more in-depth. It is observed that most of the human genes that share peptide sequences with SARS-CoV-2 belong to the, for example:
KEGG level 1 Organismal Systems (containing KEGG level 2, see table 13: environmental adaptation, and aging; and KEGG level 3: thermogenesis, circadian entrainment, circadian rhythm, longevity regulating pathway)
KEGG level 1 Human Diseases (containing KEGG level 2, see table 13: viral infectious disease, endocrine and metabolic disease, specific types cancer, neurodegenerative disease, cardiovascular disease, antineoplastic drug resistance; and KEGG level 3: herpes simplex virus 1 infection, human papillomavirus infection, human cytomegalovirus infection, human T-cell leukemia virus 1 infection, Cushing syndrome, insulin resistance, AGE-RAGE signaling pathway in diabetic complications, Huntington disease, Alzheimer disease, Parkinson disease, dilated cardiomyopathy, fluid shear stress and atherosclerosis, small cell lung cancer, breast cancer, endometrial cancer, endocrine resistance, EGFR tyrosine kinase inhibitor resistance)
KEGG level 1 Environmental Information Processing (containing KEGG level 2, see table 13: membrane transport, signal transduction; and KEGG level 3: ABC transporters, PI3K-Akt-, MAPK-, cAMP-, Calcium-, Rap1-, cGMP-PKG-, Ras-, JAK-STAT-, Phospholipase D-. AMPK-, Wnt-, Apelin-, Hippo-, mTOR-, FoxO-, HIF-1-, NF-kappa B-, Sphingolipid-, Phosphatidylinositol-, TNF-, ErbB-, TGF-beta-, Hedgehog-signaling pathway)
KEGG level 1 Cellular Processes (containing KEGG level 2, see table 13: cellular community; and KEGG level 3: focal adhesion, tight junction, gap junction, adherents junction)

The most of the increase in the number of shared peptide sequences over time is seen in KEGG level 1 Genetic Information Processing (KEGG level 2: translation, and folding, sorting and degradation), KEGG level 1 Metabolism, KEGG level 1 Environmental Information Processing and KEGG level 1 Human Diseases. Also, the physiological effects of the pathways can be retrieved from the said database, based upon which pathway-related diseases are identified by the skilled person.

In table 13, for each of the KEGG level 2, the total numbers of involved distinct human genes and the numbers of involved distinct human genes containing conserved 5-7 AA sequences shared with SARS-CoV-2 (KEGG level 4), and distinct shared peptide sequences are listed as identified in the NCBI database of April 2020 (‘2020’) and April 2021 (‘2021’). Shaded areas represent either a more than 25% of the total number of involved human genes or a more than 40% increase in the number of distinct shared peptide sequences.

It is to be understood that the above identification is a general concept, applicable to virtually all infectious diseases in any host from any pathogen. In step a., the proteome of the host and that of the pathogen are compared in order to identify conserved regions of at least 4 consecutive amino acids, and in step b., one or more pathway classes are identified from a systematic database as described above, comprising pathway classes and protein data of the host.

In accordance with the above, it is also possible to identify conserved regions of at least 4 consecutive amino acids in one or more proteins of the pathogen that are not shared by human proteins, or not shared by human proteins that play a role in one or more defined physiological pathways, preferably not shared by any human protein. Such a stretch of at least 4 consecutive amino acids can be used for the preparation of e.g. a vaccine. Therefore, in another embodiment, the medicament is a vaccine against COVID-19.

The medicament, in particular a vaccine, comprises one or more agents interacting with at least one region of at least 4 consecutive amino acids present in the SARS-CoV-2 proteome, the said at least one region of at least 4 consecutive amino acids being selected by identification of regions of at least 4 consecutive amino acids not conserved between the SARS-CoV-proteome and the human proteome; and selecting the said region of at least 4 consecutive amino acids for the preparation of the medicament or vaccine. In case of a vaccine, the said region can be e.g. used as an antigen.

TABLE 13
KEGG pathways level 2 identified as based on human conserved 5-7 AA sequences shared with SARS-CoV-2.

| | Total | # distinct human genes | | | | | # distinct shared peptide | | |
|---|---|---|---|---|---|---|---|---|---|
| | human | (KEGG Level 4) | | | | | sequences | | |
| | genes | Multiple countries | | | | | Multiple countries | | |
| | (KEGG | | | 2020- | | | | 2020- | |
| KEGG level 2 | Level 4) | 2020 | | 2021 | | % Increase | 2020 | 2021 | % Increase |
| 1.0 Global and overview maps | 1251 | 191 | (15%) | 247 | (20%) | 29% | 188 | 244 | 30% |
| 1.1 Carbohydrate metabolism | 372 | 59 | (16%) | 75 | (20%) | 27% | 60 | 77 | 28% |
| 1.2 Energy metabolism | 147 | 11 | (7%) | 15 | (10%) | 36% | 10 | 14 | 40% |
| 1.3 Lipid metabolism | 433 | 56 | (13%) | 66 | (15%) | 18% | 55 | 66 | 20% |
| 1.4 Nucleotide metabolism | 203 | 25 | (12%) | 32 | (16%) | 28% | 28 | 40 | 43% |
| 1.5 Amino acid metabolism | 286 | 51 | (18%) | 66 | (23%) | 29% | 56 | 77 | 38% |
| 1.6 Metabolism of other amino acids | 112 | 10 | (9%) | 17 | (15%) | 70% | 13 | 21 | 62% |
| 1.7 Glycan biosynthesis and metabolism | 243 | 23 | (9%) | 41 | (17%) | 78% | 27 | 46 | 70% |
| 1.8 Metabolism of cofactors and vitamins | 196 | 20 | (10%) | 25 | (13%) | 25% | 18 | 24 | 33% |
| 1.9 Metabolism of terpenoids and polyketides | 22 | 3 | (14%) | 5 | (23%) | 67% | 3 | 5 | 67% |
| 1.10 Biosynthesis of other secondary metabolites | 10 | 0 | (0%) | 0 | (0%) | 0% | 0 | 0 | 0% |
| 1.11 Xenobiotics biodegradation and metabolism | 105 | 10 | (10%) | 11 | (10%) | 10% | 9 | 10 | 11% |
| 1.12 Chemical structure transformation maps | 0 | 0 | | 0 | | 0% | 0 | 0 | 0% |
| 2.1 Transcription | 206 | 35 | (17%) | 44 | (21%) | 26% | 34 | 41 | 21% |
| 2.2 Translation | 450 | 59 | (13%) | 91 | (20%) | 54% | 62 | 106 | 71% |
| 2.3 Folding, sorting and degradation | 459 | 66 | (14%) | 96 | (21%) | 45% | 73 | 108 | 48% |
| 2.4 Replication and repair | 304 | 42 | (14%) | 51 | (17%) | 21% | 50 | 66 | 32% |
| 3.1 Membrane transport | 43 | 15 | (35%) | 16 | (37%) | 7% | 21 | 26 | 24% |
| 3.2 Signal transduction | 1686 | 345 | (20%) | 447 | (27%) | 30% | 316 | 422 | 34% |
| 3.3 Signaling molecules and interaction | 743 | 132 | (18%) | 165 | (22%) | 25% | 134 | 174 | 30% |
| 4.1 Transport and catabolism | 728 | 72 | (10%) | 112 | (15%) | 56% | 76 | 125 | 64% |
| 4.2 Cell growth and death | 993 | 92 | (9%) | 121 | (12%) | 32% | 97 | 132 | 36% |
| 4.3 Cellular community - eukaryotes | 556 | 135 | (24%) | 170 | (31%) | 26% | 137 | 179 | 31% |
| 4.4 Cellular community - prokaryotes | 0 | 0 | | 0 | | 0% | 0 | 0 | 0% |
| 4.5 Cell motility | 209 | 33 | (16%) | 47 | (22%) | 42% | 43 | 62 | 44% |
| 5.1 Immune system | 962 | 143 | (15%) | 187 | (19%) | 31% | 144 | 192 | 33% |
| 5.2 Endocrine system | 758 | 168 | (22%) | 216 | (28%) | 29% | 166 | 236 | 42% |
| 5.3 Circulatory system | 250 | 57 | (23%) | 72 | (29%) | 26% | 61 | 84 | 38% |
| 5.4 Digestive system | 376 | 93 | (25%) | 107 | (28%) | 15% | 109 | 141 | 29% |
| 5.5 Excretory system | 149 | 33 | (22%) | 35 | (23%) | 6% | 43 | 52 | 21% |
| 5.6 Nervous system | 477 | 100 | (21%) | 126 | (26%) | 26% | 116 | 148 | 28% |
| 5.7 Sensory system | 557 | 94 | (17%) | 109 | (20%) | 16% | 88 | 110 | 25% |
| 5.8 Development and regeneration | 720 | 55 | (8%) | 72 | (10%) | 31% | 70 | 90 | 29% |
| 5.9 Aging | 100 | 31 | (31%) | 33 | (33%) | 6% | 40 | 46 | 20% |
| 5.10 Environmental adaptation | 121 | 53 | (52%) | 76 | (63%) | 21% | 67 | 90 | 34% |
| 6.1 Cancer: overview | 881 | 196 | (20%) | 242 | (25%) | 23% | 192 | 247 | 29% |
| 6.2 Cancer: specific types | 400 | 94 | (24%) | 115 | (29%) | 22% | 93 | 119 | 28% |
| 6.3 Infectious disease: viral | 660 | 216 | (33%) | 280 | (42%) | 30% | 204 | 275 | 35% |
| 6.4 Infectious disease: bacterial | 464 | 64 | (14%) | 85 | (18%) | 33% | 72 | 94 | 31% |
| 6.5 Infectious disease: parasitic | 271 | 55 | (20%) | 63 | (23%) | 15% | 57 | 68 | 19% |
| 6.6 Immune disease | 307 | 30 | (10%) | 35 | (11%) | 17% | 33 | 40 | 21% |
| 6.7 Neurodegenerative disease | 338 | 60 | (18%) | 80 | (24%) | 33% | 68 | 97 | 43% |
| 6.8 Substance dependence | 282 | 43 | (15%) | 50 | (18%) | 16% | 46 | 57 | 24% |
| 6.9 Cardiovascular disease | 261 | 49 | (17%) | 62 | (22%) | 27% | 64 | 85 | 33% |
| 6.10 Endocrine and metabolic disease | 349 | 93 | (27%) | 108 | (31%) | 16% | 99 | 127 | 28% |
| 6.11 Drug resistance: antimicrobial | 0 | 0 | | 0 | | 0% | 0 | 0 | 0% |
| 6.12 Drug resistance: antineoplastic | 217 | 48 | (22%) | 55 | (25%) | 15% | 60 | 72 | 20% |
| 7.1 Chronology: Antiinfectives | 0 | 0 | | 0 | | 0% | 0 | 0 | 0% |
| 7.2 Chronology: Antineoplastics | 0 | 0 | | 0 | | 0% | 0 | 0 | 0% |
| 7.3 Chronology: Nervous system agents | 0 | 0 | | 0 | | 0% | 0 | 0 | 0% |
| 7.4 Chronlogy: Other drugs | 0 | 0 | | 0 | | 0% | 0 | 0 | 0% |
| 7.5 Target-based classification: G protein-coupled receptors | 0 | 0 | | 0 | | 0% | 0 | 0 | 0% |
| 7.6 Target-based classification: Nuclear receptors | 0 | 0 | | 0 | | 0% | 0 | 0 | 0% |
| 7.7 Target-based classification: Ion channels | 0 | 0 | | 0 | | 0% | 0 | 0 | 0% |
| 7.8 Target-based classification: Transporters | 0 | 0 | | 0 | | 0% | 0 | 0 | 0% |
| 7.9 Target-based classification: Enzymes | 0 | 0 | | 0 | | 0% | 0 | 0 | 0% |
| 7.10 Structure-based classification | 12 | 0 | (0%) | 0 | (0%) | 0% | 0 | 0 | 0% |

| | | | | | | | |
|---|---|---|---|---|---|---|---|
| 7.11 Skeleton-based classification | 0 | 0 | 0 | 0% | 0 | 0 | 0% |

KEGG Level 1
1. Metabolism
2. Genetic Information Processing
3. Environmental Information Processing
4. Cellular Processes
5. Organismal Systems
6. Human Diseases
7. Drug Development

This novel approach for explaining the clinical manifestations and complications of COVID-19 and finding of peptides for their effective treatment can thus be used for defining safe genomic and proteomic regions for the design of safe vaccines (e.g. SARS-CoV-2 vaccines) by avoiding conserved regions shared with the human genome and proteome, in order to prevent side effects.

The region of at least 4 consecutive amino acids is identified the same way as described above, however now based on the region being not conserved between the host and the pathogen, in casu human and SARS-CoV-2, respectively. The very same methodology can be used, but the identification of the region is made based on difference in sequence instead of homology to be used in the vaccine is preferably not found in the top level pathway classes of the host, more preferably not found in any of the other pathway classes. This way, a vaccine can be developed wherein the region of at least 4 consecutive amino acids can be incorporated in a synthetic peptide providing for an epitope not shared by the host.

In an attractive embodiment, the said vaccine also comprises one or more synthetic peptides identified as possible medicament for treatment of a pathway-associated disease as described above. Such a combination provides for an effective epitope as antigen for the host to elicit an immune response, as well as one or more medicaments effective against any side effect brought about by the vaccine. Such a medicament is preferably intended to be administered to a patient in need thereof in a single composition, or as separate compositions, simultaneously, or subsequently.

Again, according to the same methodology, a vaccine against virtually any infectious pathogen can be developed. In step a., the proteome of the host and that of the pathogen are compared in order to identify regions of at least 4 consecutive amino acids, that are not shared, and in step b., one or more pathway classes are identified from a systematic database as described above, comprising pathway classes and protein data of the host.

Proteome data can e.g. be obtained by sequencing the genome of the pathogen and deduct the proteins encoded by the said genome. In many cases, it is possible to obtain such proteome data from publicly available databases e.g. SWISSPROT, Uniprot (https://www.uniprot.org/), NCBI (https://www.ncbi.nlm.nih.gov/protein/), EMBL-EBI (https://www.ebi.ac.uk/), SIB (https://www.expasy.org/) and HUGO (https://www.genenames.org/). The skilled person will be aware of other databases that also can be used for the envisaged aim.

For the proteome comparison between the infectious pathogen and the host, in casu SARS-CoV-2 and human, respectively, any suitable tool known to the skilled person can be used. The proteome comparison in step a. is preferably performed by perfect matching, in particular using SIB ExPASy tools.

The systematic database comprising a plurality of physiological pathway classes as described above are preferably provided by one or more appropriate databases, in particular STRING (https://string-db.org/), Reactome (https://reactome.org), KEGG (https://www.genome.jp/kegg/), IntAct (https://www.ebi.ac.uk/intact/) and Ingenuity (https://digitalinsights.qiagen.com/). However, any other collection of databases dealing with genomes, biological pathways, biological processes, biological systems, diseases, drugs, chemical substances, symptoms databases, drugs adverse effect database, or genomic and proteomic interactions for bioinformatics research and education, including data analysis in genomics, metagenomics, metabolomics, interactomics, and other omics studies, modelling and simulation in systems biology, and translational research in drug development, known to the skilled person can be used. The KEGG pathway and the physiological pathway classification thereof are preferentially used for the identification of the conserved regions of the peptides and medicaments of the invention.

The identification of one or more physiological pathways in step b. is preferably done based on a false discovery rate of 0.05 or less, preferably of 10⁻⁴ or less, more preferably of 10⁻⁵ or less, preferably of 10⁻⁶ or less.

Preferably, in step b. the pathway identification is performed by a 100% identity comparison of the conserved consecutive amino acids with the systematic database used.

In an attractive embodiment, therapeutic peptides are selected according to a region of at least 4 consecutive amino acids is that present in one or more proteins that belong to at least 5 different pathway classes. Such a medicament will be effective in a broad range of diseases and can effectively be used against symptoms, pathologies, clinical manifestations, and complications of COVID-19.

The one or more amino acid regions preferably comprise at least 5 consecutive amino acids, or 6 consecutive amino acids, or even 7 or 8 consecutive amino acids. As explained herein, the number of identified regions decrease with increasing length of the region, but on the other hand, the longer the region, the more specific and narrower the number of identified pathways will be. A limited pathway number may be desired when a specific medicament is sought to treat a dysfunction in a pathway, where the corresponding amino acid sequence is also present in other pathways.

Peptide sequences as identified herein, relating to either a specific human pathway or group of human pathways can interfere with the host protein-protein interactions by known mechanisms, for example by orthosteric competition with the associated peptide sequence(s) in one or more proteins of the pathway(s) and/or binding or competition with allosteric sites. Such peptide sequences can be synthesized according to known procedures and used for the treatment of COVID-19 patients, and patients with other diseases with pathologies and/or complications shared with COVID-19 patients as outlined op page 2. These pharmaceuticals will also be useful for the treatment of SARS-CoV-2 infection and related pathologies.

The conserved peptide sequences identified herein can be used for the functional modulation of these pathways. Hence, the peptides claimed in this patent are not just useful for the treatment of COVID-19, but several of these are also applicable for the treatment of separate viral and non-viral diseases that share mechanisms, pathology or complications with COVID-19 e.g. (a)septic shock, organ failure, particularly for renal- and heart failure, ischemia-reperfusion injury, addiction, psychiatric disease, Alzheimer disease and related dementias, neurodegenerative diseases and for antivirals treatment.

The application of such peptide pharmaceuticals may be oral, mucosal, by i.v. or other injection or infusion, by inhalation or other means, whether or not in combination with an adjuvant or a suitable vehicle or modification for stabilization or gradual release, or with any intracellular delivery system.

Moreover, by the identified shared peptide sequences, biological processes and their pathways that play a crucial role in the clinical manifestations and complications of COVID-19 are identified. This is valuable in drug repurposing as several of the identified biological processes and their pathways can also be targeted by known drugs. This approach can also be used for finding appropriate peptides for the treatment of various manifestations of cancer, based on the analysis of the conserved parts of the genome and proteome of oncogenic viruses (e.g. HPV, HIV), and comparison with the human genome and proteome.

In the above five independent in silico approaches several peptide pharmaceuticals were identified. These in silico approaches are (1) selection based on shared SARS-CoV-2 sequences and the human complete proteome (COVID-19); (2) selection based on shared SARS-CoV-2 sequences and human proteins involved in ‘aging’-biological process (longevity/aging); (3) selection based on shared SARS-CoV-2 sequences and human proteins involved in ‘viral life cycle’-biological process (viral life cycle); (4) selection based on shared SARS-CoV-2 sequences and human proteins involved in ‘viral integration’-pathways (viral integration); (5) selection based on shared SARS-CoV-2 sequences and human proteins involved in various biological systems.

The identified peptides from the above 1 to 4 approaches in which SARS-CoV-2 sequence data was obtained from the NCBI databases in April 2020, namely based on COVID-19 and the physiological classes of longevity/aging, viral life cycle and viral integration are listed in Table 7, 14 and 15.

Table 7 shows the values of each of the pharmaceutical peptides to interfere with pathways involved in each of the four groups (1 to 4 approaches). The differential values for each of the peptides in Table 7 show their differential preference for application in COVID-19 and other viral infections. The value in the column Longevity/aging is to be taken in account dependent on the severity of the disease, e.g. the higher the value in this column the more preferred the use of the peptide in severe cases of the disease.

Table 14 gives a summary of identified peptides for the treatment of COVID-19 patients (class COVID-19), aging related diseases (class Longevity/aging) and patients with viral diseases in general (classes Viral life cycle and Viral integration) as deduced from these analyses. This Table lists the peptides to be used as pharmaceuticals for the treatment of COVID-19 and/or promote longevity/delay aging and/or to interfere with the Viral life cycle and/or to interfere with Viral integration or any of these separately.

TABLE 14
Peptides for use in COVID-19, aging and
other viral diseases

| | Pharmaceutical |
|---|---|
| Class | peptides (SEQ) |
| COVID-19, Longevity/ | AVGLE, ALLLL, LLLVA, LVLLP |
| aging, Viral life | |
| cycle, Viral | |
| integration | |
| COVID-19, Longevity/ | VAVGL, GAGAA, LGVLV, LLQAP, |
| aging, Viral life | LLVAA, VLLPL |
| cycle | |
| COVID-19, Longevity/ | VLGLA, GLHLL, SSSRS, LALGG, |
| aging, Viral | ALGVL, LPVLQV, QLPQG |
| integration | |
| COVID-19, Viral life | LLPLV, VLQAV, LVLLPL, LGGLH |
| cycle, Viral | |
| integration | |
| COVID-19, Longevity/ | VLDLL, EEEQE, AALGV, VLQLP, |
| aging | LDLLV |
| COVID-19, Viral life | SSCSS, LLVAAG, LQAVG, |
| cycle | VLPQL ,LVAAG |
| COVID-19, Viral | VLLPLV, HLLLVA, SSRSR, |
| integration | SCSSS, SSSRSR, HLLLVAA, |
| | LALLLL, VLGLAA, SSRSSS, |
| | LGGLHL, AALGVL, LALLL, |
| | RLALGG, VAVGLE, LLLVAA, |
| | SSCSSS, VLQLPQG, GGLHLL, |
| | CSSSRS, SCSSSR, SSCSSSR, |
| | VLQAVG |
| Longevity/aging, | VADGL, LVAVG, DGVDV, VLQPE, |
| Viral life cycle | AVLLA, AADPA, GLAAV, LVLLL, |
| | VGVAL, LVGVL, VVEVV, VLLLL, |
| | LLLQL, EEAAR, VAAVL, |
| | SSLSS, LLLLV |
| COVID-19 | GVLPQL, LGVLVP, VLDLLV, |
| | LPLQL, AADLOD, LQLPQ, |
| | GGLHL, PAADL, ADLDD, |
| | PAADLD, AGAAL, GAGAAL, |
| | AADLD, GVLPQ, VLQLPQ, |
| | LPLQLG |
| Longevity/aging | FIKRS, RQVLL, LDRLE, KIKTI, |
| | VLVPL, PVPEV, LGLLP |
| Viral life cycle | GDAAL, LTSFS, LHVVG, |
| | TFSSIF, FSSIF, QPGVA, |
| | EQEED, VDGQV, TFSSI, |
| | PLVEQ |
| Viral integration | IFWFS, IIFWF |

In order to select peptides from Table 7 that can be used for the treatment of COVID-19 associated pathologies as mentioned in FIG. 1, proteins can be identified from any protein(gene)-disease-associated database that is linked to the pathologies in FIG. 1. Such a database contains curated and inferred gene-disease associations. Curated protein(gene)-disease associations are extracted from the published literature. Inferred associations are established via curated chemical-gene interactions (e.g., gene A is associated with disease B because gene A has a curated interaction with chemical C, and chemical C has a curated association with disease B).

This way, peptides that are shared in the human diseases/complications (as shown in FIG. 1)—associated proteins linked to COVID-19 associated pathologies.

Table 15 shows a list of SARS-CoV-2 shared peptide sequences found in the human protein-disease database that is involved in the pathologies mentioned in FIG. 1, independent of whether these pathologies occur in the context of SARS-CoV-2/COVID-19. These shared peptide sequences can be used as pharmaceuticals for the treatment of the in FIG. 1 mentioned COVID-19 associated pathologies.

TABLE 15
SARS-COV-2 shared peptide sequences
found in human protein-disease
database related to aging and longevity

| | | | | |
|---|---|---|---|---|
| ALGVL | ALLLL | AVGLE | VAVGL | EEAAR |
| EQEED | FIKRS | GLAAV | IIFWF | LGVLV |
| LLLQL | LLLVA | LLPLV | PAADL | PVPEV |
| QLPQG | VLQLP | SRSSS | SSCSS | VDGQV |

In a following aspect, the invention relates to a method for the preparation of a medicament against a pathogen infection or against a pathology related to the said infection, the medicament comprising a synthetic peptide comprising a region comprising at least 4 consecutive amino acids, comprising the steps of:
a. identification of one or more conserved regions of at least 4 consecutive amino acids with a 100% match between the proteome of the pathogen and the proteome of the host;
b. identification of at least one pathway class from a systematic database comprising a plurality of physiological pathway classes of the said host, each class comprising a plurality of host proteins that are functionally related to the said physiological pathway, wherein the said at least one pathway class shares at least one pathology and/or complication of the infection as a result of dysfunction in the said pathway and comprises at least one host protein comprising regions of at least 4 consecutive amino acids that are conserved with a match of 100% between the pathogen proteome and the host proteome; and
c. selecting the said identified conserved region of at least 4 consecutive amino acids of step a), present in the at least one protein comprised in the pathway class identified in step b) for the preparation of the medicament.

Such a method is based on the principle disclosed herein and provides a very elegant way to determine suitable amino acid regions that can be used in synthetic peptides for the preparation of a medicament, without being bound to a single pathogen or host.

As discussed above, step a. can be replaced by steps a1 and a2,
step a1. comprising identification of one or more biological processes from a systematic database, the database comprising a plurality of biological processes of the host and, for each biological process, a plurality of host proteins that are functionally related to the said biological process, and
step a2. comprising identification of at least one conserved region of at least 4 consecutive amino acids in one or more of the plurality of proteins identified in step a1., said conserved region of at least 4 consecutive amino acids having a 100% match with the pathogen proteome.

As discussed above, step a. can be also replaced by steps a3 and a4,
step a3. comprising identification of one or more biological system from a systematic database, the database comprising a plurality of human biological systems and, for each biological system, a plurality of human pathway maps representing wiring diagrams of proteins and other gene products responsible for various cellular functions involved in a biological system, and
step a4. comprising identification of at least one conserved region of at least 4 consecutive amino acids in one or more of the plurality of proteins identified in step a3., said conserved region of at least 4 consecutive amino acids having a 100% match with the SARS-CoV-2 proteome.

Using this approach (selection on the basis of biological systems by using KEGG level 1 to 4), the following other peptide sequences were identified in the SARS-CoV-2 proteome obtained from NCBI in April 2021 for the treatment of septic shock and platelet activation, ischemia-reperfusion injury, viral infection, neuroinflammation/neurodegenerative diseases, and cardiovascular diseases (Table 16). The peptide sequences of the first two columns under “Septic Shock and Platelet Activation” represent the region of at least 5 consecutive amino acids that can preferably be used for a medicament for use in the treatment of septic and aseptic shock, or for the treatment of aberrant platelet function. The peptide sequences of the third column under “Viral infection/COVID-19” represent the region of at least 5 consecutive amino acids that can preferably be used for a medicament for use in the treatment of a viral infection including SARS-CoV-2/COVID-19, Antibody-Dependent Enhanced COVID-19 and related Multisystem Inflammatory Syndromes. The peptide sequences of the fourth column under “Cardiovascular Diseases” represent the region of at least 5 consecutive amino acids that can preferably be used for a medicament for use in the treatment of cardiac infarcts and cardiovascular diseases.

TABLE 16
List of conserved human peptide sequences
shared with SARS-COV-2

| Septic | | | | | Neuroinflammation |
|---|---|---|---|---|---|
| Shock and | | Viral | Ischemia | Cardio- | and Neuro |
| Platelet | | infection/ | Reperfusion | vascular | degenerative |
| Activation | | COVID-19 | Injury | Diseases | Diseases |
| AALGV | LLLQL | ALREL | AALGV | ALVLL | AVGVV |
| AALGVL | LLLVAA | ARLDP | ALGVL | CISTK | GSKCRSK |
| ALGVL | LLALV | AVGLE | ALREL | DPPEA | FSRIL |
| AVGLE | LLVLG | CFNCL | AVGLE | HGLPG | GAGAA |
| AVGVV | LVLVA | DLQQL | AVGVV | LGVLV | KRFKK |
| AVLGV | LVPAV | EAVEA | AVLLA | LHALV | LALGG |
| AVLGVL | LVPHV | GEVLV | GAGAA | LALLL | LLLDQ |
| AVLLA | LVPLV | GLPQG | GDAAL | LLLLV | LRIIT |
| DGDPD | LVPQE | HAAVD | GEVLV | LQLEE | NITSF |
| ELGVL | NIFLS | HARAR | HGLPG | LQQLR | SCSSS |
| ELGVY | PLGVE | IKNEK | KIKTI | LVGVL | VEQDD |
| ERLVP | PVPEV | INFTI | LGVGG | PEAEV | VVALL |
| GDAAL | QPVDLVP | KSNII | LGVLY | PPEAE | |
| HLGVD | RELGV | LARLD | LALLE | PVPEV | |
| ILIST | RIKIL | LHLLL | LHVVG | RLDPP | |
| KIKTI | SLVPG | LLVLV | LLLDQ | SIYNL | |
| LGVEL | RQVLL | LVRGL | LLLQL | SSPTIK | |
| LGVELE | SSCSS | RGLPQ | LLLVAA | SSYII | |
| LGVGG | TSSSK | SRSRS | LLPLV | VAAGL | |
| LGVLH | VADGL | SSRSR | LLVLG | VEAEV | |
| LGVLV | VALLV | VELVY | LLVLV | | |
| LGVLVP | VDLVP | VLLLL | PVPEV | | |
| LGVVH | VLGVL | VRGLA | QPVDLVP | | |
| LHLLL | VLPQL | WNSNK | RIKIL | | |
| LHVVG | VLVPH | LLLVA | RCIVLL | | |
| LLGVG | VLVPHY | VLPQL | SCSSS | | |
| LLGVGG | VLVPL | ALGVL | TSSSK | | |
| VLLLLVAV | VVEVV | TFSSIF | VEQDD | | |
| LLPAADLDD | | FSSIF | VLGVL | | |
| GAGAAL | | AVGVV | VLPQL | | |
| LGVLVP | | GAGAA | VLVPL | | |
| LVLLPLV | | LALGG | VVALL | | |
| LLGVGG | | AVGVV | VVEVV | | |
| GVLPQLEQ | | VADGL | VLLLLVAV | | |
| AALGVLVPL | | LVPAV | LLPAADLDD | | |
| | | VLVPL | GAGAAL | | |
| | | AALGV | LGVLVP | | |
| | | VLLLLVAV | LVLLPLV | | |
| | | LLPAADLDD | LLGVGG | | |
| | | GAGAAL | GVLPQLEQ | | |
| | | LGVLVP | AALGVLVPL | | |
| | | LVLLPLV | | | |
| | | LLGVGG | | | |
| | | GVLPQLEQ | | | |
| | | AALGVLYPL | | | |

The peptide sequences of the fifth column under “Neuro-inflammation and Neurodegenerative diseases” represent the region of at least 5 consecutive amino acids that can preferably be used for a medicament for use in the treatment of neuroinflammation and neurodegeneration.

The conserved regions of table 16 were obtained by analysis starting from biological systems for treatment of (septic) shock, inhibition of platelet activation, inhibition of viral infection, treatment of ischemia reperfusion injury, treatment of cardiovascular diseases, treatment of neurodegenerative diseases/neuro-inflammatory diseases. Two peptides (DGDPD, VALLV) are variants not shared by SARS-CoV-2.

In the following embodiment, the invention relates to a method for the preparation of a host vaccine against pathogen infection, the vaccine comprising a synthetic peptide or comprising an RNA-sequence with a region comprising or coding for at least 4 consecutive amino acids, comprising the steps of:
a. identification of at least one region of at least 4 consecutive amino acids not conserved between the pathogen proteome and the host proteome; the said at least one region of at least 4 consecutive amino acids not being present in one or more proteins belonging to at least one pathway class from a systematic database comprising a plurality of physiological pathway classes of the host, each class comprising a plurality of host proteins that are functionally related to the said physiological pathway;
b. selecting the said region of at least 4 consecutive amino acids for the preparation of the peptide or RNA vaccine.

As indicated above, the method as described herein can conveniently be used for the identification of suitable conserved peptide sequences and the preparation of synthetic peptides comprising such conserved peptide sequences, without being bound to a single pathogen or host. The vaccine can also be supplemented and comprise one or more region of at least 4 consecutive amino acids conserved between the pathogen proteome and the host proteome as identified earlier approaches 1 to five in order to reduce side effects.

The above analyses are done starting from sequence identity between the conserved human proteome and SARS-CoV-2 proteome and selecting conserved sequences based on pathways, biological processes, and/or biological systems. The conserved peptide sequences can also be selected for individual organs or groups of organs, tissue compartments, cell lineages, and subcellular levels. The Human Protein Atlas (https://www.proteinatlas.org/; Uhlen M et al., Tissue-based map of the human proteome. Science (2015): PubMed: 25613900) provides data about the enhanced expression of distinct genes in these different compartments. All, approximately 20,000, human genes are classified according to their expression across all major organs and tissue types in the human body. Few of the genes are strictly tissue-specific, however, the genes with an elevated expression in particular tissues are interesting as a starting point to understand their biology and function, and the underlying mechanisms for disease, and for defining biomarkers for diagnosis and prognosis of disease, and prediction of adverse events of a therapeutic compound.

Transcriptome analysis of all major organs and tissue types in the human body can be visualized with regard to specificity and distribution of transcribed mRNA molecules across all putative 19,670 protein-coding genes, the analysis includes 11,069 genes and 8385 genes with low tissue specificity (the housekeeping proteome). Elevated expression includes three subcategory types of elevated expression:
Tissue enriched: At least four-fold higher mRNA level in a particular tissue compared to any other tissue.
Group enriched: At least four-fold higher average mRNA level in a group of 2-5 tissues compared to any other tissue.
Tissue enhanced: At least four-fold higher mRNA level in a particular tissue compared to the average level in all other tissues.

A total of 11,069 genes are elevated in at least one of the analysed tissues of which: 2845 are tissue enriched genes, 1637 are group enriched genes, and 6587 are tissue enhanced genes.

Distribution, on the other hand, visualizes how many genes have, or do not have, detectable levels (NX_1) of transcribed mRNA molecules. All elevated genes are categorized as:
Detected in single: Detected in a single tissue
Detected in some: Detected in more than one but less than one-third of tissues
Detected in many: Detected in at least a third but not all tissues
Detected in all: Detected in all tissues

Starting at this point the inventors determined the expression of conserved human peptide sequences shared with SARS-CoV-2 using the NCBI database of April 2021. When comparing various organs an extreme presence of such sequences was found in the RNA of brain-expressed genes, followed by the testis, skeletal muscle, lymphoid tissue, intestine, liver, blood, and heart muscle (Table 17). At the tissue level, the expression of such sequences was especially prominent in neuronal cells (bipolar cells, cone photoreceptor cells, horizontal cells, and rod photoreceptor cells), germ cells (early spermatids, late spermatids, spermatocytes, Muller glia cells, and spermatogonia), cardiomyocytes, ciliated cells, hepatocytes, Kupffer cells, enterocytes, trophoblast cells and alveolar cells (Table 18). Within the brain, the expression of genes containing conserved human peptide sequences shared with SARS-CoV-2 was especially prominent in the pons and medulla, the cerebellum, and the basal ganglia (Table 19). This data parallels the clinical picture of diverse COVID-19 associated pathologies, not only affecting the respiratory system but at earlier stage of the disease pathology involving many other organs, especially the brain and the peripheral functions of the brain (amongst others respiration), intestine, heart, and testis. This allows a more specific organ-directed selection of therapeutic peptides as outlined above by the inventors. It also allows the selection of peptides for (local) therapeutic application, e.g. dispersion in the lungs.

At the subcellular level (Table 20) the conserved human peptide sequences shared with the SARS-CoV-2 proteome especially showed up in the nucleoplasm, the cytosol, the vesicles, the plasma membrane, the mitochondria, and the Golgi apparatus.

The conserved human peptide sequences shared with SARS-CoV-2 were found to occur in all different blood cell lineages (Table 21), but especially in granulocytes and T-cells, and least expressed in NK cells. With regards to the different cell types, especially the expression of genes with human-SARS-CoV-2 conserved sequences in neutrophilic, basophilic, and eosinophilic granulocytes, and plasmacytoid dendritic cells was prominent, confirming the prominent role of these blood cell types in the development of the pathologies and complications of COVID-19 (Table 21), and therefore also of diagnostic value.

This is also true for the secreted gene products that contain the conserved human peptide sequences shared with SARS-CoV-2 that can be detected in blood and other tissue fluids (Tables 21 and 22). The increased intracellular and membrane expression of these sequences, and the secreted products in blood and the extracellular matrix allow the automated detection of these products for diagnostic and prognostic purposes.

TABLE 17
Transcriptome analysis of all major organs.

| | #distinct genes | | | #distinct sequences | | |
|---|---|---|---|---|---|---|
| | Tissue | Tissue | Group | Tissue | Tissue | Group |
| | enhanced | enriched | enriched | enhanced | enriched | enriched |
| adipose tissue | 30 | 0 | 8 | 34 | 0 | 10 |
| adrenal gland | 31 | 0 | 14 | 37 | 0 | 17 |
| blood | 123 | 9 | 48 | 137 | 12 | 58 |
| bone marrow | 67 | 5 | 14 | 95 | 7 | 21 |
| brain | 358 | 112 | 107 | 433 | 133 | 129 |
| breast | 9 | 4 | 8 | 11 | 5 | 9 |
| cervix, uterine | 20 | 0 | 6 | 23 | 0 | 9 |
| ductus deferens | 17 | 0 | 5 | 21 | 0 | 5 |
| endometrium 1 | 13 | 0 | 3 | 19 | 0 | 5 |
| epididymis | 37 | 7 | 16 | 49 | 7 | 19 |
| esophagus | 44 | 1 | 12 | 56 | 1 | 16 |
| fallopian tube | 40 | 3 | 20 | 52 | 4 | 25 |
| gallbladder | 21 | 0 | 9 | 26 | 0 | 11 |
| heart muscle | 49 | 8 | 41 | 82 | 10 | 48 |
| intestine | 104 | 26 | 39 | 123 | 34 | 59 |
| kidney | 43 | 9 | 25 | 58 | 11 | 29 |
| liver | 95 | 41 | 45 | 116 | 47 | 58 |
| lung | 33 | 4 | 14 | 41 | 4 | 17 |
| lymphoid tissue | 196 | 17 | 44 | 239 | 19 | 61 |
| ovary | 31 | 0 | 4 | 42 | 0 | 5 |
| pancreas | 48 | 9 | 16 | 54 | 9 | 21 |
| parathyroid gland | 45 | 5 | 7 | 60 | 6 | 8 |
| pituitary gland | 51 | 7 | 23 | 68 | 8 | 33 |
| placenta | 68 | 17 | 16 | 83 | 21 | 19 |
| prostate | 21 | 2 | 3 | 27 | 2 | 4 |
| retina | 37 | 18 | 12 | 51 | 24 | 15 |
| salivary gland | 43 | 8 | 12 | 55 | 8 | 19 |
| seminal vesicle | 23 | 1 | 6 | 25 | 1 | 6 |
| skeletal muscle | 118 | 29 | 54 | 160 | 35 | 71 |
| skin 1 | 45 | 11 | 23 | 59 | 14 | 33 |
| smooth muscle | 21 | 0 | 4 | 32 | 0 | 6 |
| stomach 1 | 16 | 2 | 10 | 19 | 3 | 12 |
| testis | 170 | 173 | 66 | 221 | 214 | 87 |
| thyroid gland | 71 | 8 | 11 | 92 | 10 | 12 |
| tongue | 36 | 3 | 39 | 60 | 3 | 55 |
| urinary bladder | 14 | 0 | 5 | 19 | 0 | 6 |
| vagina | 8 | 0 | 3 | 12 | 0 | 3 |

In table 17, the number of different human genes containing the number of SARS-CoV-2 conserved sequences with elevated expression in three subcategory types (tissue enhanced, tissue enriched, group enriched) are shown.

TABLE 18
Transcriptome analysis at a the tissue level (single-cell level)

| | | #distinct | #distinct |
|---|---|---|---|
| | | genes | sequences |
| Blood & immune | B-cells | 41 | 51 |
| cells | Erythroid cells | 51 | 75 |
| | granulocytes | 73 | 87 |
| | Hofbauer cells | 83 | 91 |
| | Kupffer cells | 97 | 109 |
| | Macrophages | 66 | 76 |
| | monocytes | 67 | 79 |
| | T-cells | 61 | 70 |
| Endocrine cells | Intestinal endocrine cells | 71 | 84 |
| | Leydig cells | 58 | 69 |
| | Pancreatic endocrine cells | 47 | 56 |
| Epithelial cells | Alveolar cells type 1 | 80 | 109 |
| | Alveolar cells type 2 | 88 | 115 |
| | Basal glandular cells | 27 | 33 |
| | Basal keratinocytes | 85 | 110 |
| | Cholangiocytes | 56 | 77 |
| | Ciliated cells | 165 | 222 |
| | Club cells | 71 | 95 |
| | Collecting duct cells | 54 | 70 |
| | Distal tubular cells | 54 | 66 |
| | Ductal cells | 38 | 46 |
| | Enterocytes | 115 | 142 |
| | Exocrine glandular cells | 33 | 37 |
| | Glandular cells | 83 | 99 |
| | Hepatocytes | 152 | 189 |
| | Mucus-secreting cells | 71 | 83 |
| | Paneth cells | 78 | 94 |
| | Proximal tubular cells | 110 | 138 |
| | Sertoli cells | 77 | 97 |
| | Suprabasal keratinocytes | 66 | 83 |
| | Urothelial cells | 42 | 50 |
| Germ cells | Early spermatids | 474 | 598 |
| | Late spermatids | 391 | 492 |
| | Muller glia cells | 143 | 184 |
| | Spermatocytes | 198 | 251 |
| | Spermatogonia | 101 | 136 |
| Mesenchymal cells | Fibroblasts | 33 | 40 |
| | Ito cells | 69 | 86 |
| | Peritubular cells | 79 | 96 |
| Muscle cells | Cardiomyocytes | 172 | 235 |
| | Smooth muscle cells | 41 | 54 |
| Neuronal cells | Bipolar cells | 208 | 270 |
| | Cone photoreceptor cells | 197 | 275 |
| | Horizontal cells | 202 | 258 |
| | Rod photoreceptor cells | 158 | 224 |
| Pigment cells | Melanocytes | 48 | 61 |
| Trophoblast cells | Cytotrophoblasts | 67 | 91 |
| | Extravillous trophoblasts | 92 | 121 |
| | Syncytiotrophoblasts | 100 | 123 |
| Undifferentiated | Undifferentiated cells | 48 | 54 |
| cells | | | |
| Vascular cells | Endothelial cells | 30 | 31 |

In Table 18, the number of different human genes containing the number of SARS-CoV-2 conserved sequences with elevated expression in a tissue cell is shown.

TABLE 19
Transcriptome analysis of the brain

| | #distinct | #distinct |
|---|---|---|
| | genes | sequences |
| amygdala | 10 | 10 |
| basal ganglia | 43 | 54 |
| cerebellum | 71 | 97 |
| hypothalamus | 15 | 15 |
| cerebral cortex | 25 | 26 |
| midbrain | 6 | 8 |
| hippocampal formation | 13 | 14 |
| pons and medulla | 80 | 101 |
| thalamus | 20 | 21 |
| olfactory region | 1 | 1 |

In table 19, the number of different human genes containing the number of SARS-CoV-2 conserved sequences with elevated expression in various brain tissue is shown.

TABLE 20
Transcriptome analysis at the subcellular level.

| | Subcellular | #distinct | #distinct |
|---|---|---|---|
| | main location | genes | sequences |
| Nucleus | Nuclear membrane | 11 | 14 |
| | Nucleoli | 17 | 20 |
| | Nucleoli fibrillar center | 14 | 16 |
| | Nucleoli rim | 2 | 6 |
| | Nucleoplasm | 243 | 250 |
| | Kinetochore | 0 | 0 |
| | Mitotic chromosome | 0 | 0 |
| | Nuclear bodies | 15 | 17 |
| | Nuclear speckles | 16 | 17 |
| Cytoplasm | Actin filaments | 17 | 21 |
| | Cleavage furrow | 0 | 0 |
| | Focal adhesion sites | 3 | 3 |
| | Intermediate filaments | 9 | 12 |
| | Centrosome | 19 | 25 |
| | Centriolar satellite | 6 | 7 |
| | Microtubules | 15 | 22 |
| | Cytokinetic bridge | 0 | 0 |
| | Microtubule ends | 0 | 0 |
| | Midbody | 1 | 1 |
| | Midbody ring | 1 | 1 |
| | Mitotic spindle | 1 | 2 |
| | Cytosol | 140 | 148 |
| | Aggresome | 0 | 0 |
| | Cytoplasmic bodies | 4 | 4 |
| | Rods & Rings | 0 | 0 |
| | Mitochondria | 67 | 76 |
| Secretory | Endoplasmic reticulum | 19 | 23 |
| (without | Golgi apparatus | 57 | 67 |
| secreted | Vesicles | 124 | 135 |
| proteins) | Endosomes | 0 | 0 |
| | Lipid droplets | 0 | 0 |
| | Lysosomes | 0 | 0 |
| | Peroxisomes | 1 | 1 |
| | Plasma membrane | 109 | 133 |
| | Cell Junctions | 18 | 24 |
| Secretory | Intracellular and membrane | 203 | 223 |
| Proteins | Secreted - unknown location | 26 | 31 |
| (secretom | Secreted in brain | 15 | 16 |
| [symbol] | Secreted in female | 5 | 6 |
| | reproductive system | | |
| | Secreted in male | 9 | 10 |
| | reproductive system | | |
| | Secreted in other tissues | 43 | 55 |
| | Secreted to blood | 110 | 116 |
| | Secreted to digestive system | 12 | 12 |
| | Secreted to extracellular matrix | 79 | 94 |

[symbol]

indicates data missing or illegible when filed

In table 20, the number of different human genes containing the number of SARS-CoV-2 conserved sequences with elevated expression in various subcellular main locations are shown.

TABLE 21
Transcriptome analysis in different
blood cell lineages and their cells

| | #distinct | #distinct |
|---|---|---|
| | genes | sequences |
| B-cells | 105 | 131 |
| dendritic cells | 176 | 213 |
| granulocytes | 275 | 345 |
| monocytes | 152 | 179 |
| NK-cells | 56 | 70 |
| T-cells | 240 | 295 |
| basophil | 134 | 172 |
| classical monocyte | 131 | 148 |
| eosinophil | 94 | 117 |
| gdT-cell | 60 | 68 |
| intermediate monocyte | 93 | 104 |
| MAIT T-cell | 59 | 68 |
| memory B-cell | 66 | 82 |
| memory CD4 T-cell | 47 | 59 |
| memory CD8 T-cell | 47 | 53 |
| myeloid DC | 74 | 82 |
| naive B-cell | 79 | 99 |
| naive CD4 T-cell | 68 | 87 |
| naive CD8 T-cell | 53 | 63 |
| neutrophil | 189 | 238 |
| NK-cell | 63 | 77 |
| non-classical monocyte | 81 | 93 |
| plasmacytoid DC | 137 | 167 |
| T-reg | 83 | 105 |

In table 21, the number of different human genes containing the number of SARS-CoV-2 conserved sequences with elevated expression in various blood cell lineages and blood cells are shown.

Viral infection involves a large number of genomic and protein interactions, including protein-protein interactions between the virus and its host. These interactions range from the initial binding of viral coat proteins to host membrane receptors to the hijacking of the host transcription and metabolic machinery by viral proteins. It seems that a successful virus knows its host minimum targets to defeat the host defence systems. Amino acid sequence similarity over the larger region between virus and host is relatively low, so sequence-based prediction of virus-host interactions, especially for new viruses or hosts is challenging. Herein, it is shown that identifying the relatively smaller regions between a virus and its host helps to understand the mechanism of viral infections, transmission, and the spectrum of clinical manifestations, and to design appropriate treatment.

For example:
1. Recent data indicate the prevalence of variants with N440K Spike substitution in several parts of India, which is under the second wave of the pandemic. It has been predicted that the N440K variant could lead to a virus higher rate of transmission (Tandel et al. N440K variant of SARS-CoV-2 has higher infectious fitness. BioRxiv). The analyses of the inventors show that in the case of N440 the Spike protein region ‘WNSNNLK’ has no similarity with human conserved peptide sequences, while after N440K substitution the ‘WNSNK’ peptide sequence is shared by human late secretory pathway protein AVL9 homolog (AVL9_Human/AVL9 gene). According to the gene ontology annotations (GO annotation), AVL9 protein is active in the cytoplasm. It is located in the membrane, as an integral component of membrane, endosome, and recycling endosome. AVL9 is involved in cell migration, membrane trafficking, and autophagy in budding yeast.
2. Yet another example is T7161 substitution in the UK variant with higher transmission. The analyses of the inventors show that in this case the Spike protein region ‘TNFTIV’ has no similarity with human protein sequences, while TNFTI peptide is shared by human neural recognition molecule Contactin-5 (CNTN5 gene). After T7161 substitution ‘INFTI’ peptide sequence is shared by human trafficking protein particle complex subunit 8 (TPPC8_Human/TRAPPC8 gene). According to the GO annotation, TPPC8 protein plays a role in the endoplasmic reticulum to Golgi apparatus trafficking at a very early stage (PubMed:21525244), maintains together with TBC1D14 the cycling pool of ATG9 required for initiation of autophagy (PubMed:26711178), and acts as a host factor required for human papillomavirus cell entry (PubMed: 24244674).
3. Yet another example is D215G substitution in the ZA (South Africa) variant. The analyses of the inventors show that in this case the Spike protein region ‘LVRDL’ is present in the human component of the histone deacetylase NuRD complex (chromodomain-helicase-binding protein 3; CHD3_Human/CHD3 gene) which according to GO annotation participates in the remodelling of chromatin by deacetylating histones (PubMed:9804427, PubMed:30397230), is involved in transcriptional repression as part of the NuRD complex (PubMed:27068747), is required for anchoring centrosomal pericentrin in both interphase and mitosis, and for spindle organization and centrosome integrity (PubMed:17626165). After D215G substitution no similarity is found with CHD3 protein, while peptides ‘LVRGL’, ‘GLPQG’, ‘RGLPQ’ and ‘VRGLP’ originate that are present in innate defence response interferon regulatory factor 7 protein (IRF7_Human/IRF7 gene), antigen peptide transporter 1 protein (TAP1_Human/TAP1 gene), E3 ubiquitin-protein ligase Jade-2 protein (JADE2_Human/JADE2 gene), neurotransmitter transporter (S6A17_Human, S6A17 gene), and cilia- and flagella-associated protein 69 (CFA69_Human/CFA69 gene), NUDT2, CRIP3, FAM135B, MAP7D2, SGSM1, TAP1, SLC22A31, TBX19, CC2D1A, and LIMD1. All these proteins are involved in various important biological processes relating to virus recognition, innate immune response, virus clearance to neuro-cellular differentiation, olfaction, and sensory transduction.
4. Yet another example is P681H substitution in the UK variant. The analyses of the inventors show that in this case the Spike protein region ‘PRRAR’ which is present in human protein NK1 transcription factor-related protein 2 (NKX1-2 gene). After P681H substitution peptide ‘PRRAR’ disappear and peptide ‘HRRAR’ originate which is present in human sodium/hydrogen exchanger 6 protein (SLC9A6 gene) and human Myb/SANT-like DNA-binding domain-containing protein 1 (MSANTD1 gene).
5. Yet another example is S982A substitution in the UK variant. The analyses of the inventors show that in this case the Spike protein has no similarity in this region with human proteins. After S982A substitution peptides ‘ARLDP and ‘LARLD’ originate that are present in human protein VPS9 domain-containing protein 1 (VPS9D1 gene), human protein Leucine-rich repeat and fibronectin type III domain-containing protein 3 (LRFN3 gene), human protein Nesprin-2 (SYNE2 gene), and human protein THO complex subunit 5 homolog (THOC5 gene).
6. Yet another example is the K986P and V986P substitutions found in the core region of BioNTech/Pfizer spike protein mRNA vaccine on purpose to enhance the vaccine efficiency. The analysis of the inventors shows that due to K986P and V986P substitutions the shared peptide sequence ‘VEAEV’ disappeared which is found in AHNAK2, GMCL1, MYH7B, SMC4, TBL1XR1 and USP10 human genes. However, due to K986P and V986P substitutions the shared peptide sequences ‘DPPEA’, ‘PEAEV’, ‘PPEAE’ and ‘RLDPP’ originate. These conserved sequences are present in the human genes DNAH2, CD276, EPB41L5, RPGRIP1, RUNX2, TTN, ANO7, HSF1, HYDIN, KIAA1614, SF11, TTC28, CACNB1, CACNG1, CACNG5, CACNG8, ITGA5, ITGA10, KCNH2, RUNX2, TGFB1, and TTN. These genes are part of human pathways (KEGG, Reactome) related to inflammation, hypertrophic cardiomyopathy, dilated cardiomyopathy, arrhythmogenic right ventricular cardiomyopathy, cardiac conduction, phase 0-rapid depolarisation, phase 2-plateau phase, cardiac muscle contraction, muscle contraction, adrenergic signalling in cardiomyocytes, oxytocin signalling pathway, and RUNX2 regulation of genes involved in cell migration.

The above method for selection of therapeutic peptides was also applied on the proteome of Human Papilloma Virus (HPV). In the proteomes of 218 of the known 222 HPV strains together 347 unique conserved peptide sequences were found that also occur in the collection of 1704 unique conserved peptide sequences found in the SARS-CoV-2 proteome (Table 3). 6 of these were identical to peptides that in the experiments below proved effective in the inhibition of SARS-CoV-2 replication (namely SRSRS, LLVLV, SSRSR, VLLLL, DLQQL, LHLLL), 15 were identical to peptides that proved effective in the treatment of septic shock and platelet activation (namely RQVLL, VLQLP, LVPHV, QLPQG, LHLLL, PVPEV, LLPLV, LGVGG, SRSSS, AVLLA, TSSSK, LVLVP, ILIST, VAVGL, LLLQL), 7 were identical to peptides that were effective in the treatment of cardiovascular problems (namely LLLV, LHLLL, PVPEV, LQQLR, SIYNL, LQLEE, LHALV), and 1 was identical to a peptide that was effective in the treatment of neurodegeneration (namely GAGAA). In the analysis of HPV proteomes also a clear distinction was found between the HPV strains HPV-6, HPV-16 and HPV-18 with regard to the collection of unique conserved peptide sequences that are part of KEGG Level 3 pathways. Also with regard to these pathways a large overlap was found in unique conserved peptides identified in the proteomes of SARS-CoV-2 and HPV-16 and HPV-18. These data underline the value of this approach for the development of therapeutic peptides for other applications, such as cancer or any other disease.

The invention also relates to a method of treatment of COVID-19 comprising the step of administering the medicament or therapeutic composition as described herein to a patient in need thereof. Such patient is in particular a mammal, more in particular a human.

The invention will be further explained by way of the appended figures and following examples.

FIG. 1: Scheme of the sequence of (patho)physiological events leading to various pathological conditions of which the outcome is determined by the genes or their (by)products related to longevity pathways.

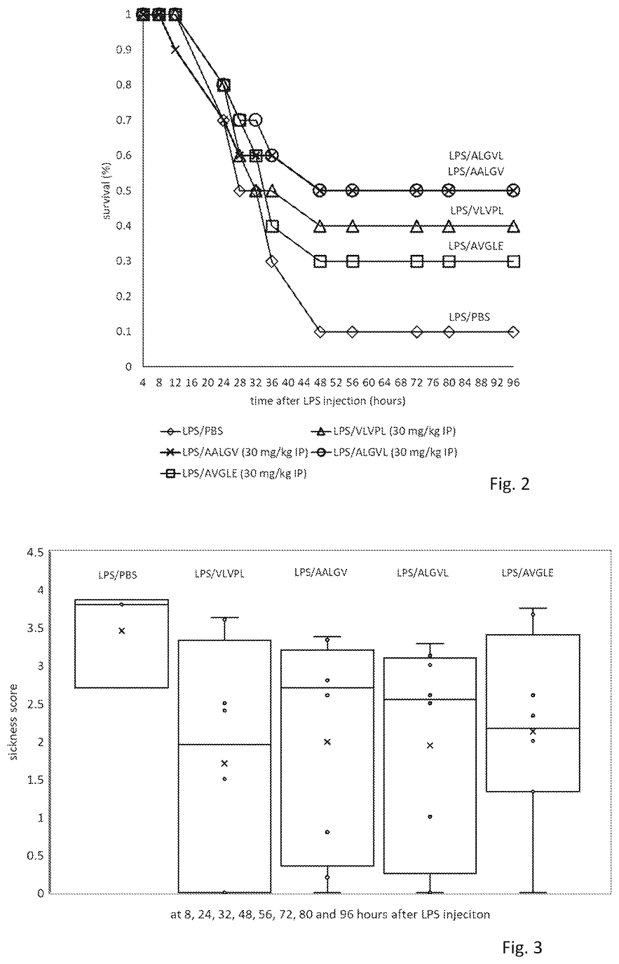

FIG. 2: FIG. 2. Survival of BALB/c mice after i.p. injection of 20 mg/kg E. coli O111:B4 LPS and treated with 30 mg/kg peptide 30 minutes after LPS administration (n=10 per group).

FIG. 3: Sickness scores of the mice of the experiment shown in FIG. 2 at 8, 24, 32, 48, 56, 72, 80, and 96 hours after LPS injection.

### EXAMPLES

In vitro as well as in vivo experiments support the treatment efficacy of the selected peptide pharmaceuticals in various important aspects of COVID-19 and its related pathologies.

### Peptide Synthesis

Peptides were prepared commercially by solid-phase synthesis using the fluorenylmethoxycarbonyl (Fmoc)/tert-butyl-based methodology with 2-chlorotrityl chloride resin as the solid support. The side-chain of glutamine was protected with a trityl function. The peptides were synthesized manually. Each coupling consisted of the following steps: (i) removal of the alpha-amino Fmoc-protection by piperidine in dimethylformamide (DMF), (ii) coupling of the Fmoc amino acid (3 eq) with diisopropylcarbodiimide (DIC)/1-hydroxybenzotriazole (HOBt) in DMF/N-methylformamide (NMP) and (iii) capping of the remaining amino functions with acetic anhydride/diisopropylethylamine (DIEA) in DMF/NMP. Upon completion of the synthesis, the peptide resin was treated with a mixture of trifluoroacetic acid (TFA)/H2O/triisopropylsilane (TIS) 95:2.5:2.5. After 30 minutes TIS was added until decolorization. The solution was evaporated in vacuo and the peptide precipitated with diethyl ether. The crude peptides were dissolved in water (50-100 mg/ml) and purified by reverse-phase high-performance liquid chromatography (RP-HPLC). HPLC conditions were: column: Vydac TP21810C18 (10×250 mm); elution system: gradient system of 0.1% TFA in water v/v (A) and 0.1% TFA in acetonitrile (ACN) v/v (B); flow rate 6 ml/min; absorbance was detected from 190-370 nm. There were different gradient systems used for different peptides. Ultimately, the collected fractions were concentrated to about 5 ml by rotation film evaporation under reduced pressure at 40° C. The remaining TFA was exchanged against acetate by eluting two times over a column with anion exchange resin (Merck II) in acetate form. The elute was concentrated and lyophilized in 28 hours. Peptides later were prepared for use by dissolving them in PBS.

Inhibition of SARS-CoV-2 Replication in Human Pulmonary Epithelial Cells by Selected Peptides Human conducting airway epithelial cells (Calu-3 cells or other appropriate airway epithelial cells) were cultured on Transwell membranes for more than 10 days (according to Tseng et al., J. Virol. 79:9470-9, 2005) in order to generate cultures of highly polarized cells connected by tight junctions and microvilli. Cultures were then inoculated with SARS-CoV-2 via the apical surface. Groups of 40 cultures were set up. Five (control) groups were not supplemented with peptide, the other groups were each supplemented with one of the following peptides: AVGLE, ALREL, GEVLV, LLLVAA, LLPLV, SRSRS, LLVLV, SSRSR, EAVEA, CFNCL, VLLLL, DLQQL, KSNII, HAAVD, LHLLL, VELVV, IKNFK, LLLVA, VLPQL, ALGVL, TFSSIF, TFSSI, FSSIF, AVGVV, GAGAA, LALGG, VADGL, LVPAV, VLPVL, AALGV, ARLDP, GLPQG, HRRAR, INFTI, LARLD, VLRGL, RGLPQ, VRGLP, WNSNK, VLLLVAV, LLPAADLDD, GAGAAL, LGVLVP, LVLLPLV, LLGVGG, GVLPQLEQ and AALGVLVPL (each peptide in a final concentration in culture of 10 ug/ml).

At 10, 24, 34, and 48 hrs after inoculation the apical washes were harvested from 10 cultures of each group, and the live virus titres were determined on VeroE6 cells. All tested peptides reduced the live virus titres at 24, 34 and 48 hrs after inoculation of the cultures, while the control groups did not.

### Inhibition of SARS-CoV-2 Infection of Human PBMC by Selected Peptides

Infection of short term human PBMC cultures with SARS-CoV-2 is an accepted model for studying the molecular events underlying COVID-19 (Pontelli et al., Biorxiv.org, Aug. 7, 2020). In this model PBMC cultured together with SARS-CoV-2 and supplemented with various peptides (10 ug/ml) the cellular signalling pathways were analysed by gene array. Control cultures a\were not supplemented with peptide. The following peptides were evaluated: AVGLE, ALREL, GEVLV, LLLVA, VLPQL, ALGVL, TFSSIF, TFSSI and FSSIF.

The results show that these peptides especially affect the expression of genes that are modified by the SARS-CoV-2, resulting in the change in the activity of various pathways, such as insulin signalling pathway, glutamatergic synapse, platelet activation and apelin signalling pathway.

Inhibition of LPS and TSST-1 induced COVID-19 related pathology by selected peptides E. coli strain 026:B6 LPS (‘endotoxin’ from Sigma) and TSST-1 (‘exotoxin’) induced shock in mice are widely accepted models for human sepsis and related pathology (Group 1a and Group 1b in FIG. 1). Similar pathology is frequently seen in severe cases of COVID-19. In the LPS model the systemic effects are largely mediated by macrophages, while in the TSST-1 model the systemic effects are mediated by T cells and antigen-presenting cells. In both models the systemic shock is due to an excessive inflammatory cytokine release (so-called cytokine storm) accompanied by endogenous mediators, including oxidative stress mediators, leading to characteristic pathophysiological reactions such as fever, leukopenia, thrombocytopenia, hemodynamic changes and disseminated intravascular coagulation as well as leukocyte infiltration and inflammation of various organs, all of which can ultimately lead to death (Khan et al., Clin Exp Immunol 160, 466-78, 2010).

In the LPS model, 8-10 weeks old BALB/c mice were injected intraperitoneally (i.p.) with 10 mg/kg LPS in saline. Control mice received saline i.p. only. In the TSST-1 model, 8-10 weeks old BALB/c mice were injected with 25 mg/kg D-Galactosamine in saline i.p. On the same day the latter mice are injected with 8 mcg TSST-1 subcutaneously (s.c.). Control groups are injected with either TSST-1 s.c. or D-Galactosamine i.p. only.

In both models, mice (n=8) were pre-treated with peptide (10 mg/kg) 2 hrs before injection of either LPS or D-Galactosamine/TSST-1 or treated 12 hrs after injection of either of these shock-inducing agents. In both models, sickness development is semi-quantitatively measured as described earlier (Khan et al., Hum Immunol 62, 1-7, 2002).

All peptides of Table 15 (ALGVL, ALLLL, AVGLE, VAVGL, EEAAR, EQEED, FIKRS, GLAAV, IIFWF, LGVLV, LLLQL, LLLVA, LLPLV, PAADL, PVPEV, QLPQG, VLQLP, SRSSS, SSCSS, and VDGQV) reduced septic shock and mortality in both models as compared to mice not treated with peptide, but the peptides ALGVL, ALLLL, LLPLV, EEAAR, LLLQL, VAVGL, VLQLP reduced the clinical features of the septic shock more than the other peptides.

Determination of the inflammatory mediators TNF-α and IL-6 showed that the latter peptides were also more effective in reducing the release of these mediators than the other peptides.

Since E. coli strain 026:B6 LPS from Sigma is relatively weak endotoxin (10,000 endotoxin units/mg), the ability of the other selected peptides to suppress LPS induced shock in male BALB/c mice was evaluated with E. coli 0111::B4 LPS (500,000 endotoxin units/mg) from Sigma. In this model a dose of 20 mg/kg LPS i.p., 10 mice per group, were used and the mice were treated with the selected peptides (30 mg/kg i.p.) 30 min after LPS administration. Not only the mortality overtime as evaluated carefully, but also the sickness scores of the mice. As an example of the results from the many candidate therapeutic peptides that were identified in this model and tested in the described assay the results with the least potent peptides VLVPL, AALGV, ALGVL and AVGLE as shown in FIG. 2. These peptides significantly reduced the LPS induced mortality with 20% or more. All other selected peptides tested, and listed in Table 16 (LLVLG, LHLLL, AVLLA, ALGVL, AALGV, PVPEV, KIKTI, VLVPL, AVGVV, RQVLL, NTFLS, LHVVG, ILIST, SSCSS, GDAAL, VLPQL, RIKIL, TSSSK, AVGLE, LLLQL, LLLVAA, VVEVV, LLPLV, VADGL, AALGVL, AVLGV, AVLGVL, ELGVL, ELGVV, HLGVD, LGVEL, LGVELE, LGVGG, LGVLH, LGVLV, LGVLVP, LGVVH, LLGVG, LLGVGG, PLGVE, RELGV, VLGVL, ERLVP, LVLVP, LVPAV, LVPHV, LVPLV, LVPQE, QPVDLVP, RLVPG, VDLVP, VLVPH, VLVPHV, VLLLVAV, LLPAADLDD, GAGAAL, LGVLVP, LVLLPLV, GVLPQLEQ and AALGVLVPL) were at least as effective as the four peptides VLVPL, AALGV, ALGVL and AVGLE in inhibiting the LPS induced mortality in this model using 20 mg/kg of this highly potent endotoxin. Next to the 20 peptides of Table 15 and these 55 selected conserved human peptide sequences (Table 16) also occurring in SARS-CoV-2, two related human peptide sequences not occurring in SARS-CoV-2 were evaluated in the latter shock model, namely VALLV and DGDPD. These two peptides were also successful in inhibiting septic shock in this mouse model.

All these peptides not only significantly reduced the LPS induced mortality, but also the clinical signs of septic shock, such as reduced curiosity and locomotor activity, followed by piloerection, shivering, laboured breathing, and lethargy. The reduction of the initial clinical signs of reduced curiosity of the LPS treated mice by the peptide treatment suggests that the peptides may also reduce the neuroinflammation caused by the high dose of LPS (FIG. 3).

### Amelioration of Neuroinflammation by Selected Peptides

LPS injection in mice can induce a variety of neuroinflammatory and neurodegenerative pathologies including Parkinson's disease and other degenerative brain diseases (Deng et al., Brain, Behavior and Immunity—Health 4:1-12, 2020). The ability of selected peptides to reduce neuroinflammation and neurodegeneration was confirmed in male C57BL mice injected with LPS. In this model the mice (n=12) are i.p. injected with 10 mg/kg E. coli 0111::B4 LPS in saline or, as a control, with saline only. At 6 hrs after LPS injection 6 mice per group are killed, their brains removed and homogenized in lysis buffer. In the supernatant of the brain extracts of the LPS injected mice the TNFalpha and IL-1 beta levels were increased at least four-fold compared to the control mice.

In parallel locomotion studies were done with the other 6 mice of both groups of (conditioned) mice and their activity and behaviour recorded for two minutes every 4 hours for a total of 24 hrs. The LPS injected mice showed a sharp drop of locomotor activity 4 hrs after the LPS injection which gradually restored to about 50% of the control mice at 24 hrs.

Treatment of separate groups of similar treated mice with one of the following peptides AVGVV, FSRIL, GAGAA, SCSSS, NITSF, LLLDQ, CSKCRSK, LALGG, KRFKK, LRIIT, VEQDD and VVALL (30 mg/kg i.p. immediately after injection of either LPS or saline) reduced the TNFalpha and IL-1 beta levels in the brain extracts of the LPS injected mice 4 and 6 hrs after LPS injection with more than 50% as compared to the control mice, and significantly restored the locomotor activity of the LPS injected mice.

### Amelioration of Ischemia-Reperfusion Injury by Selected Peptides

The peptides ALGVL, EEAAR, IIFWF, GLAAV, SSCSS, VAVGL, QLPQG, VLQLP, AVGLE, LLVLV, AVGVV, GAGAA, SCSSS, LLLDQ, VEQDD, VVALL, LLLVLG, LHLLL, AVLLA, AALGV, ALREL, GEVLV, PVPEV, KIKTI, RQVLL, LHVVG, GDAAL, VLPQL, RIKIL, TSSSK, LLLQL, LLLVAA, LLPLV, LGVGG, LGVLV, VVEVV, VLGVL, QPVDLVP, HGLPG, VLLLVAV, LLPAADLLD, GAGAAL, LGVLVP, LVLLPLV, LLGVGG, GVLPQLEQ and AALGVLVPL (10 mg/kg BWV) inhibit the ischemia-reperfusion injury in 12-16 weeks old C57BL mice. In this model, the mice were anaesthetized by isoflurane inhalation, and the left renal pedicle clamped for 25 minutes using an atraumatic micro-vascular clamp as described before (Khan et al., Nephrol Dial Transplant 24, 2701-8, 2009). After release of the clamp, restoration of blood flow was inspected visually, and a contra-lateral nephrectomy was performed. The abdominal wound was closed, and the mice were given rest to recover for 3 days. After 3 days, survival of control mice (n=10) not treated with a peptide is 50%, while mice (n=10 per peptide) treated with either one of the above mentioned peptides (10 mg/kg) survive 100%. This model is generally accepted as a representative model for the evaluation of the effectiveness of candidate pharmaceuticals for use in the treatment of other reperfusion-ischemia pathologies as listed in Group 3 of FIG. 1.

### Inhibition of Platelet Activation by Selected Peptides

The effect of selected peptides on platelet activation was determined using a standard flow cytometric assay based on thrombin-induced human platelet activation. Fluorescent antibodies were used to quantify the activated platelet surface glycoprotein P-selectin to assess the extent of inhibition. In this assay the very same 61 peptides were tested as in the septic shock assay. All these peptides were separately assayed at a concentration of 50 ug/mL, and at this concentration all these peptides significantly inhibited the platelet activation in this assay.

### Inhibition of Myocardial Infarction Mice by Selected Peptides

The effect of selected peptides on myocardial infarction was determined in a standard C57BL/6 mouse model (Lim et al. Cardiovasc. Pathol. 13:91-97, 2004). Mice (n=10 per group), 10-12 weeks old, were anaesthetized by 1.5% halothane and maintained with 1% halothane. Ischaemia was induced by filamentous ligation of the left anterior descending coronary artery. Peptide (20 mg/kg BVW) was administered 1 hr before ligation. The extent of myocardial infarction was hemodynamically and histologically determined in a standardized manner at various days after ligation. The following selected peptides proved to reduce the size of the myocardial infarction in this model: HGLPG, LGVLV, SSYII, VAAGL, LHALV, CISTK, SSFTIK, LLLLV, LQLEE, LQQLR, LVGVL, SIYNL, ALVLL, LHLLL, PVPEV, DPPEA, PEAEV, PPEAE, RLDPP and VEAEV.

1. Medicament for use in the treatment of COVID-19 and related pathologies, the said medicament comprising or interacting with one or more conserved regions of at least 4 consecutive amino acids present with a 100% match in both the SARS-CoV-2 proteome and the human proteome.

2. Medicament of claim 1, wherein the one or more conserved regions of at least 4 consecutive amino acids with a 100% match are selected by:
a. identifying one or more conserved regions of at least 4 consecutive amino acids with a 100% match between the SARS-CoV-2 proteome and the human proteome;
b. identifying at least one pathway class from a systematic database comprising a plurality of human physiological pathway classes, each class comprising a plurality of human proteins that are functionally related to the said physiological pathway, wherein the said at least one pathway class shares at least one pathology and/or complication of the COVID-19 infection as a result of dysfunction in the said pathway and comprises at least one human protein comprising one or more conserved regions of at least 4 consecutive amino acids that have a 100% match with the SARS-CoV-2 proteome; and
c. selecting the said identified one or more conserved regions of at least 4 consecutive amino acids of step a., present in the at least one protein comprised in the pathway class identified in step b. for the preparation of the medicament.

3. Medicament of claim 2, wherein step a. is replaced by steps a1. and a2.,
a1. identifying one or more biological processes from a systematic database, the database comprising a plurality of human biological processes and, for each biological process, a plurality of human proteins that are functionally related to the said biological process, and
a2. identifying at least one conserved region of at least 4 consecutive amino acids in one or more of the plurality of proteins identified in step a1., said conserved region of at least 4 consecutive amino acids having a 100% match with the SARS-CoV-2 proteome.

4. Medicament of claim 1, being a vaccine against COVID-19, the said medicament comprising one or more agents interacting with at least one region of at least 4 consecutive amino acids present in the SARS-CoV-2 proteome, the said region of at least 4 consecutive amino acids being selected by:
a. identifying at least one region of at least 4 consecutive amino acids not conserved between the SARS-CoV-2 proteome and the human proteome; and
b. selecting the said at least one region of at least 4 consecutive amino acids for the preparation of the vaccine.

5. Medicament of claim 4, wherein step a. comprises:
a. identifying at least one region of at least 4 consecutive amino acids not conserved between the SARS-CoV-2 proteome and the human proteome; the said at least one region of at least 4 consecutive amino acids not being present in one or more proteins belonging to at least one pathway class from a systematic database comprising a plurality of physiological pathway classes, each class comprising a plurality of proteins that are functionally related to the said physiological pathway.

6. Medicament of claim 4, further comprising a medicament comprising or interacting with one or more conserved regions of at least 4 consecutive amino acids present with a 100% match in both the SARS-CoV-2 proteome and the human proteome, wherein the medicament is intended to be administered to a patient in need thereof in a single composition, or as separate compositions, simultaneously, or subsequently.

7. Medicament of claim 1, wherein:
a. the proteome data for step a. are obtained or derived from a database, chosen from SWISSPROT, Uniprot, NCBI, EMBL-EBI, SIB and HUGO, and/or
b. the proteome comparison in step a. is performed by perfect matching, in particular using SIB ExPASy tools.

8. Medicament of claim 2, wherein:
a. the systematic database comprising a plurality of physiological pathway classes is chosen from the group, consisting of: STRING, Interactome, KEGG, IntAct and Ingenuity, preferably KEGG; and/or
b. the physiological pathway is identified by a false discovery rate of 0.05 or less, preferably of 10-4 or less, more preferably of 10-5 or less, preferably of 10-6 or less.

9. Medicament of claim 2, wherein
a. the pathway identification in step b. is performed by a 100% identity comparison of the conserved consecutive amino acids with the systematic database, and/or
b. the region of at least 4 consecutive amino acids is present in one or more proteins that belong to at least 5 different pathway classes.

10. Medicament of claim 1, wherein the one or more conserved amino acid regions comprise:
a. at least 5 consecutive amino acids; or
b. 4 to 8 consecutive amino acids.

11. Medicament of claim 10, wherein the region of at least 5 consecutive amino acids is chosen from the group, consisting of: ALGVL, ALLLL, AVGLE, EEAAR, EQEED, FIKRS, GAGAA, GLAAV, GLHLL, IIFWF, LALGG, LGGLH, LGVLV, LLLQL, LLLVA, LPVLQV, LLPLV, LLQAV, LLVAA, LVLLPL, PAADL, PVPEV, QLPQG, SRSSS, SSCSS, SSSRS, VDGQV, LVLLP, VAVGL, VLGLA, VLLPL, VLQAV, VLQLP, VLDLL, EEEQE, AALGV, LDLLV, LLVAAG, LQAVG, VLPQL, LVAAG, VLLPLV, HLLLVA, SSRSR, SCSSS, SSSRSR, HLLLVAA, LALLLL, VLGLAA, SSRSSS, LGGLHL, AALGVL, LALLL, RLALGG, VAVGLE, LLLVAA, SSCSSS, VLQLPQG, GGLHLL, CSSSRS, SCSSSR, SSCSSSR, VLQAVG, VADGL, LVAVG, DGVDV, VLQPE, AVLLA, AADPA, LVLLL, VGVAL, LVGVL, VVEVV, VLLLL, VAAVL, SSLSS, LLLLV, GVLPQL, LGVLVP, VLDLLV, LPLQL, AADLDD, LQLPQ, GGLHL, ADLDD, PAADLD, AGAAL, GAGAAL, AADLD, GVLPQ, VLQLPQ, LPLQLG, RQVLL, LDRLE, KIKTI, VLVPL, LGLLP, GDAAL, LTSFS, LHVVG, TFSSIF, FSSIF, QPGVA, TFSSI, PLVEQ, IIFWFS, HGLPG, SSYII, VAAGL, LHALV, CISTK, SSFTIK, LQLEE, LQQLR, SIYNL, ALVLL, LHLLL, DPPEA, PEAEV, PPEAE, RLDPP, LLVLV, AVGVV, LLLDQ, VEQDD, VVALL, LLLVLG, ALREL, GEVLV, RIKIL, TSSSK, LGVGG, VLGVL, QPVDLVP, AALGV, AALGVL, AVGLE, AVGVV, AVLGV, AVLGVL, AVLLA, ELGVL, ELGVV, ERLVP, GDAAL, HLGVD, ILIST, KIKTI, LGVEL, LGVELE, LGVGG, LGVLH, LGVLV, LGVLVP, LGVVH, LHLLL, LHVVG, LLGVG, LLGVGG, LLLQL, LLLVAA, LLPLV, LLVLG, LVLVP, LVPAV, LVPHV, LVPHV, LVPLV, LVPQE, NTFLS, PLGVE, PVPEV, QPVDLVP, RELGV, RIKIL, RLVPG, RQVVL, SSCSS, TSSSK, VADGL, VALLV, VDLVP, VLGVL, VLPQL, VLVPH, VLVPHV, VLVPL, VVEVV, VLLLLVAV, LLPAADLDD, GAGAAL, LGVLVP, LVLLPLV, LLGVGG, GVLPQLEQ, AALGVLVPL, VALLV and DGDPD.

12. Medicament of claim 11 for use in the treatment of a viral infection including SARS-CoV-2/COVID-19, Antibody-Dependent Enhanced COVID-19 and related Multisystem Inflammatory Syndromes, wherein the region of at least 5 consecutive amino acids is chosen from the group, consisting of AVGLE, ALREL, GEVLV, LLLVAA, LLPLV, SRSRS, LLVLV, SSRSR, EAVEA, CFNCL, VLLLL, DLQQL, KSNII, HAAVD, LHLLL, VELVV, IKNFK, LLLVA, VLPQL, ALGVL, TFSSIF, TFSSI, FSSIF, AVGVV, GAGAA, LALGG, VADGL, LVPAV, VLVPL, VLLLLVAV, LLPAADLDD, GAGAAL, LGVLVP, LVLLPLV, LLGVGG, GVLPQLEQ, AALGVLVPL and AALGV.

13. Medicament of claim 11 for use in the treatment or prevention of septic and aseptic shock, or for the treatment of aberrant platelet function, wherein the region of at least 5 consecutive amino acids is chosen from the group, consisting of ALGVL, ALLLL, AVGLE, VAVGL, EEAAR, EQEED, FIKRS, GLAAV, IIFWF, LGVLV, LLLQL, LLLVA, LLPLV, PAADL, PVPEV, QLPQG, VLQLP, SRSSS, SSCSS, VDGQV, LLVLG, LHLLL, AVLLA, ALGVL, AALGV, KIKTI, VLVPL, AVGVV, RQVLL, NTFLS, LHVVG, ILIST, GDAAL, VLPQL, RIKIL, TSSSK, LLLVAA, VVEVV, VADGL, AALGVL, AVLGV, AVLGVL, ELGVL, ELGVV, HLGVD, LGVEL, LGVELE, LGVGG, LGVLH, LGVLVP, LGVVH, LLGVG, LLGVGG, PLGVE, RELGV, VLGVL, ERLVP, LVLVP, LVPAV, LVPHV, LVPLV, LVPQE, QPVDLVP, RLVPG, VDLVP, VLVPH, VLVPHV, AVLGV, AVLGVL, ELGVL, ELGVV, ERLVP, HLGVD, ILIST, LGVEL, LGVELE, LGVVH, LHVVG, LLGVG, LLGVGG, LLVLG, RLVPG, VADGL, VDLVP, VLVPH, VLVPHV, VLVPL, VLLLLVAV, LLPAADLDD, GAGAAL, LGVLVP, LVLLPLV, LLGVGG, GVLPQLEQ, AALGVLVPL, VALLV and DGDPD.

14. Medicament of claim 11 for use in the prevention and treatment of neuroinflammation and neurodegeneration, wherein the region of at least 5 consecutive amino acids is chosen from the group, consisting of AVGVV, FSRIL, GAGAA, SCSSS, NITSF, LLLDQ, CSKCRSK, LALGG, KRFKK, LRIIT, VEQDD and VVALL.

15. Medicament of claim 11 for use in the prevention and treatment of ischemia-reperfusion injury, wherein the region of at least 5 consecutive amino acids is chosen from the group, consisting of ALGVL, EEAAR, IIFWF, GLAAV, SSCSS, VAVGL, QLPQG, VLQLP, AVGLE, LLVLV, AVGVV, GAGAA, SCSSS, LLLDQ, VEQDD, VVALL, LLLVLG, LHLLL, AVLLA, AALGV, ALREL, GEVLV, PVPEV, KIKTI, RQVLL, LHVVG, GDAAL, VLPQL, RIKIL, TSSSK, LLLQL, LLLVAA, LLPLV, LGVGG, VVEVV, VLGVL, QPVDLVP, HGLPG, VLLLLVAV, LLPAADLDD, GAGAAL, LGVLVP, LVLLPLV, LLGVGG, GVLPQLEQ, AALGVLVPL and LGVLV.

16. Medicament of claim 11 for use in the prevention and treatment of cardiac infarcts and cardiovascular diseases, wherein the region of at least 5 consecutive amino acids is chosen from the group, consisting of HGLPG, LGVLV, SSYII, VAAGL, LHALV, CISTK, SSFTIK, LLLLV, LQLEE, LQQLR, LVGVL, SIYNL, ALVLL, LHLLL, PVPEV, DPPEA, PEAEV, PPEAE, RLDPP and VEAEV.

17. Medicament of claim 1, wherein the COVID-19 related diseases are chosen from ischemia, reperfusion, hypoxia, shock, hypovolemia, sepsis, septic shock, inflammation, heart failure, infarction, cardiac, liver and kidney failure and injury, severe acute respiratory syndrome, respiratory distress syndrome, acute lung injury, multiple organ failure, metabolic syndrome, acute or chronic heart, kidney, lung and liver disease, acute or chronic inflammatory disease, longevity and aging diseases or symptoms related to or characteristic for these diseases.

18. Method for the preparation of a medicament against a pathogen infection or against a pathology related to the said infection, the medicament comprising a synthetic peptide comprising a region comprising at least 4 consecutive amino acids, are selected by the steps of:
a. identifying one or more conserved regions of at least 4 consecutive amino acids with a 100% match between the proteome of the pathogen and the proteome of the host;
b. identifying at least one pathway class from a systematic database comprising a plurality of physiological pathway classes of the said host, each class comprising a plurality of host proteins that are functionally related to the said physiological pathway, wherein the said at least one pathway class shares at least one pathology and/or complication of the infection as a result of dysfunction in the said pathway and comprises at least one host protein comprising regions of at least 4 consecutive amino acids that are conserved with a match of 100% between the pathogen proteome and the host proteome; and
c. selecting the said identified conserved region of at least 4 consecutive amino acids of step a), present in the at least one protein comprised in the pathway class identified in step b) for the preparation of the medicament.

19. Method of claim 18, wherein step a. is replaced by steps a1 and a2,
a1. identifying one or more biological processes from a systematic database, the database comprising a plurality of biological processes of the host and, for each biological process, a plurality of host proteins that are functionally related to the said biological process, and
a2. identifying at least one conserved region of at least 4 consecutive amino acids in one or more of the plurality of proteins identified in step a1., said conserved region of at least 4 consecutive amino acids having a 100% match with the pathogen proteome.

20. Method for the preparation of a host vaccine against a pathogen infection, the vaccine comprising a peptide comprising a region comprising at least 4 consecutive amino acids, selected by the steps of:
a. identifying at least one region of at least 4 consecutive amino acids not conserved between the pathogen proteome and the host proteome; the said at least one region of at least 4 consecutive amino acids not being present in one or more proteins belonging to at least one pathway class from a systematic database comprising a plurality of physiological pathway classes of the host, each class comprising a plurality of host proteins that are functionally related to the said physiological pathway;
b. selecting the said region of at least 4 consecutive amino acids for the preparation of the vaccine.

21. Method for treatment of COVID-19 comprising administering the medicament of claim 1 to a patient in need thereof.


SEQUENCE LISTING (207 sequences)

SEQ ID NO: 1 - protein, 5 aa, Homo sapiens
AADLD

SEQ ID NO: 2 - protein, 6 aa, Homo sapiens
AADLDD

SEQ ID NO: 3 - protein, 5 aa, Homo sapiens
AADPA

SEQ ID NO: 4 - protein, 5 aa, Homo sapiens
AALGV

SEQ ID NO: 5 - protein, 6 aa, Homo sapiens
AALGVL

SEQ ID NO: 6 - protein, 5 aa, Homo sapiens
ADLDD

SEQ ID NO: 7 - protein, 5 aa, Homo sapiens
AGAAL

SEQ ID NO: 8 - protein, 5 aa, Homo sapiens
ALGVL

SEQ ID NO: 9 - protein, 5 aa, Homo sapiens
ALLLL

SEQ ID NO: 10 - protein, 5 aa, Homo sapiens
AVGLE

SEQ ID NO: 11 - protein, 5 aa, Homo sapiens
AVLLA

SEQ ID NO: 12 - protein, 6 aa, Homo sapiens
CSSSRS

SEQ ID NO: 13 - protein, 5 aa, Homo sapiens
DGVDV

SEQ ID NO: 14 - protein, 5 aa, Homo sapiens
EEAAR

SEQ ID NO: 15 - protein, 5 aa, Homo sapiens
EEEQE

SEQ ID NO: 16 - protein, 5 aa, Homo sapiens
EQEED

SEQ ID NO: 17 - protein, 5 aa, Homo sapiens
FIKRS

SEQ ID NO: 18 - protein, 5 aa, Homo sapiens
FSSIF

SEQ ID NO: 19 - protein, 5 aa, Homo sapiens
GAGAA

SEQ ID NO: 20 - protein, 6 aa, Homo sapiens
GAGAAL

SEQ ID NO: 21 - protein, 5 aa, Homo sapiens
GDAAL

SEQ ID NO: 22 - protein, 5 aa, Homo sapiens
GGLHL

SEQ ID NO: 23 - protein, 6 aa, Homo sapiens
GGLHLL

SEQ ID NO: 24 - protein, 5 aa, Homo sapiens
GLAAV

SEQ ID NO: 25 - protein, 5 aa, Homo sapiens
GLHLL

SEQ ID NO: 26 - protein, 5 aa, Homo sapiens
GVLPQ

SEQ ID NO: 27 - protein, 6 aa, Homo sapiens
GVLPQL

SEQ ID NO: 28 - protein, 6 aa, Homo sapiens
HLLLVA

SEQ ID NO: 29 - protein, 7 aa, Homo sapiens
HLLLVAA

SEQ ID NO: 30 - protein, 5 aa, Homo sapiens
IIFWF

SEQ ID NO: 31 - protein, 6 aa, Homo sapiens
IIFWFS

SEQ ID NO: 32 - protein, 5 aa, Homo sapiens
KIKTI

SEQ ID NO: 33 - protein, 5 aa, Homo sapiens
LALGG

SEQ ID NO: 34 - protein, 5 aa, Homo sapiens
LALLL

SEQ ID NO: 35 - protein, 6 aa, Homo sapiens
LALLLL

SEQ ID NO: 36 - protein, 5 aa, Homo sapiens
LDLLV

SEQ ID NO: 37 - protein, 5 aa, Homo sapiens
LDRLE

SEQ ID NO: 38 - protein, 5 aa, Homo sapiens
LGGLH

SEQ ID NO: 39 - protein, 6 aa, Homo sapiens
LGGLHL

SEQ ID NO: 40 - protein, 5 aa, Homo sapiens
LGLLP

SEQ ID NO: 41 - protein, 5 aa, Homo sapiens
LGVLV

SEQ ID NO: 42 - protein, 6 aa, Homo sapiens
LGVLVP

SEQ ID NO: 43 - protein, 5 aa, Homo sapiens
LHVVG

SEQ ID NO: 44 - protein, 5 aa, Homo sapiens
LLLLV

SEQ ID NO: 45 - protein, 5 aa, Homo sapiens
LLLQL

SEQ ID NO: 46 - protein, 5 aa, Homo sapiens
LLLVA

SEQ ID NO: 47 - protein, 6 aa, Homo sapiens
LLLVAA

SEQ ID NO: 48 - protein, 5 aa, Homo sapiens
LLPLV

SEQ ID NO: 49 - protein, 5 aa, Homo sapiens
LLQAP

SEQ ID NO: 50 - protein, 5 aa, Homo sapiens
LLVAA

SEQ ID NO: 51 - protein, 6 aa, Homo sapiens
LLVAAG

SEQ ID NO: 52 - protein, 5 aa, Homo sapiens
LPLQL

SEQ ID NO: 53 - protein, 6 aa, Homo sapiens
LPLQLG

SEQ ID NO: 54 - protein, 6 aa, Homo sapiens
LPVLQV

SEQ ID NO: 55 - protein, 5 aa, Homo sapiens
LQAVG

SEQ ID NO: 56 - protein, 5 aa, Homo sapiens
LQLPQ

SEQ ID NO: 57 - protein, 5 aa, Homo sapiens
LTSFS

SEQ ID NO: 58 - protein, 5 aa, Homo sapiens
LVAAG

SEQ ID NO: 59 - protein, 5 aa, Homo sapiens
LVAVG

SEQ ID NO: 60 - protein, 5 aa, Homo sapiens
LVGVL

SEQ ID NO: 61 - protein, 5 aa, Homo sapiens
LVLLL

SEQ ID NO: 62 - protein, 5 aa, Homo sapiens
LVLLP

SEQ ID NO: 63 - protein, 6 aa, Homo sapiens
LVLLPL

SEQ ID NO: 64 - protein, 5 aa, Homo sapiens
PAADL

SEQ ID NO: 65 - protein, 6 aa, Homo sapiens
PAADLD

SEQ ID NO: 66 - protein, 5 aa, Homo sapiens
PLVEQ

SEQ ID NO: 67 - protein, 5 aa, Homo sapiens
PVPEV

SEQ ID NO: 68 - protein, 5 aa, Homo sapiens
QLPQG

SEQ ID NO: 69 - protein, 5 aa, Homo sapiens
QPGVA

SEQ ID NO: 70 - protein, 6 aa, Homo sapiens
RLALGG

SEQ ID NO: 71 - protein, 5 aa, Homo sapiens
RQVLL

SEQ ID NO: 72 - protein, 5 aa, Homo sapiens
SCSSS

SEQ ID NO: 73 - protein, 6 aa, Homo sapiens
SCSSSR

SEQ ID NO: 74 - protein, 5 aa, Homo sapiens
SSCSS

SEQ ID NO: 75 - protein, 6 aa, Homo sapiens
SSCSSS

SEQ ID NO: 76 - protein, 7 aa, Homo sapiens
SSCSSSR

SEQ ID NO: 77 - protein, 5 aa, Homo sapiens
SSLSS

SEQ ID NO: 78 - protein, 5 aa, Homo sapiens
SSRSR

SEQ ID NO: 79 - protein, 6 aa, Homo sapiens
SSRSSS

SEQ ID NO: 80 - protein, 5 aa, Homo sapiens
SSSRS

SEQ ID NO: 81 - protein, 6 aa, Homo sapiens
SSSRSR

SEQ ID NO: 82 - protein, 5 aa, Homo sapiens
TFSSI

SEQ ID NO: 83 - protein, 6 aa, Homo sapiens
TFSSIF

SEQ ID NO: 84 - protein, 5 aa, Homo sapiens
VAAVL

SEQ ID NO: 85 - protein, 5 aa, Homo sapiens
VADGL

SEQ ID NO: 86 - protein, 5 aa, Homo sapiens
VAVGL

SEQ ID NO: 87 - protein, 6 aa, Homo sapiens
VAVGLE

SEQ ID NO: 88 - protein, 5 aa, Homo sapiens
VDGQV

SEQ ID NO: 89 - protein, 5 aa, Homo sapiens
VGVAL

SEQ ID NO: 90 - protein, 5 aa, Homo sapiens
VLDLL

SEQ ID NO: 91 - protein, 6 aa, Homo sapiens
VLDLLV

SEQ ID NO: 92 - protein, 5 aa, Homo sapiens
VLGLA

SEQ ID NO: 93 - protein, 6 aa, Homo sapiens
VLGLAA

SEQ ID NO: 94 - protein, 5 aa, Homo sapiens
VLLLL

SEQ ID NO: 95 - protein, 5 aa, Homo sapiens
VLLPL

SEQ ID NO: 96 - protein, 6 aa, Homo sapiens
VLLPLV

SEQ ID NO: 97 - protein, 5 aa, Homo sapiens
VLPQL

SEQ ID NO: 98 - protein, 5 aa, Homo sapiens
VLQAV

SEQ ID NO: 99 - protein, 6 aa, Homo sapiens
VLQAVG

SEQ ID NO: 100 - protein, 5 aa, Homo sapiens
VLQLP

SEQ ID NO: 101 - protein, 6 aa, Homo sapiens
VLQLPQ

SEQ ID NO: 102 - protein, 7 aa, Homo sapiens
VLQLPQG

SEQ ID NO: 103 - protein, 5 aa, Homo sapiens
VLQPE

SEQ ID NO: 104 - protein, 5 aa, Homo sapiens
VLVPL

SEQ ID NO: 105 - protein, 5 aa, Homo sapiens
VVEVV

SEQ ID NO: 106 - protein, 9 aa, Unknown
  SARS-CoV-2
LLPAADLDD

SEQ ID NO: 107 - protein, 9 aa, Unknown
  SARS-COV-2
SSCSSSRSR

SEQ ID NO: 108 - protein, 9 aa, Unknown
  SARS-COV-2
SSRSSSRSR

SEQ ID NO: 109 - protein, 7 aa, Unknown
  SARS-COV-2
LVLLPLV

SEQ ID NO: 110 - protein, 6 aa, Unknown
  SARS-COV-2
GAGAAL

SEQ ID NO: 111 - protein, 6 aa, Unknown
  SARS-COV-2
VLPPLL

SEQ ID NO: 112 - protein, 6 aa, Unknown
  SARS-COV-2
GGVAGA

SEQ ID NO: 113 - protein, 6 aa, Unknown
  SARS-COV-2
AALGVL

SEQ ID NO: 114 - protein, 6 aa, Unknown
  SARS-COV-2
DPAQLP

SEQ ID NO: 115 - protein, 6 aa, Unknown
  SARS-COV-2
LLGVGG

SEQ ID NO: 116 - protein, 6 aa, Unknown
  SARS-COV-2
LGVLVP

SEQ ID NO: 117 - protein, 7 aa, Unknown
  SARS-COV-2
VLQAVGA

SEQ ID NO: 118 - protein, 8 aa, Unknown
  SARS-COV-2
GVLPQLEQ

SEQ ID NO: 119 - protein, 7 aa, Unknown
  SARS-COV-2
VLQLPQG

SEQ ID NO: 120 - protein, 6 aa, Unknown
  SARS-COV-2
ALPQRH

SEQ ID NO: 121 - protein, 7 aa, Unknown
  SARS-COV-2
ALQLEEE

SEQ ID NO: 122 - protein, 6 aa, Unknown
  SARS-COV-2
LPLQLG

SEQ ID NO: 123 - protein, 6 aa, Unknown
  SARS-COV-2
VVADAV

SEQ ID NO: 124 - protein, 6 aa, Unknown
  SARS-COV-2
RLALGG

SEQ ID NO: 125 - protein, 6 aa, Unknown
  SARS-COV-2
LGGLHL

SEQ ID NO: 126 - protein, 7 aa, Unknown
  SARS-COV-2
ADGLEAP

SEQ ID NO: 127 - protein, 7 aa, Unknown
  SARS-COV-2
QPEEEQE

SEQ ID NO: 128 - protein, 9 aa, Unknown
  SARS-COV-2
LLLVAAGLE

SEQ ID NO: 129 - protein, 9 aa, Unknown
  SARS-COV-2
LLLVAAVLE

SEQ ID NO: 130 - protein, 9 aa, Unknown
  SARS-COV-2
LLLVAVGLE

SEQ ID NO: 131 - protein, 9 aa, Unknown
  SARS-COV-2
LLLVAVVLE

SEQ ID NO: 132 - protein, 8 aa, Unknown
  SARS-COV-2
VLLLLVAA

SEQ ID NO: 133 - protein, 8 aa, Unknown
  SARS-COV-2
VLLLLVAV

SEQ ID NO: 134 - protein, 8 aa, Unknown
  SARS-COV-2
VLDLLVAA

SEQ ID NO: 135 - protein, 8 aa, Unknown
  SARS-COV-2
VLDLLVAV

SEQ ID NO: 136 - protein, 5 aa, Homo sapiens
AVGVV

SEQ ID NO: 137 - protein, 5 aa, Homo sapiens
AVLGV

SEQ ID NO: 138 - protein, 6 aa, Homo sapiens
AVLGVL

SEQ ID NO: 139 - protein, 5 aa, Homo sapiens
DGDPD

SEQ ID NO: 140 - protein, 5 aa, Homo sapiens
ELGVL

SEQ ID NO: 141 - protein, 5 aa, Homo sapiens
ELGVV

SEQ ID NO: 142 - protein, 5 aa, Homo sapiens
ERLVP

SEQ ID NO: 143 - protein, 5 aa, Homo sapiens
HLGVD

SEQ ID NO: 144 - protein, 5 aa, Homo sapiens
ILIST

SEQ ID NO: 145 - protein, 5 aa, Homo sapiens
LGVEL

SEQ ID NO: 146 - protein, 6 aa, Homo sapiens
LGVELE

SEQ ID NO: 147 - protein, 5 aa, Homo sapiens
LGVLH

SEQ ID NO: 148 - protein, 5 aa, Homo sapiens
LGVVH

SEQ ID NO: 149 - protein, 5 aa, Homo sapiens
LHLLL

SEQ ID NO: 150 - protein, 5 aa, Homo sapiens
LLVLG

SEQ ID NO: 151 - protein, 5 aa, Homo sapiens
LVLVP

SEQ ID NO: 152 - protein, 5 aa, Homo sapiens
LVLVP

SEQ ID NO: 153 - protein, 5 aa, Homo sapiens
LVPAV

SEQ ID NO: 154 - protein, 5 aa, Homo sapiens
LVPHV

SEQ ID NO: 155 - protein, 5 aa, Homo sapiens
LVPLV

SEQ ID NO: 156 - protein, 5 aa, Homo sapiens
LVPQE

SEQ ID NO: 157 - protein, 5 aa, Homo sapiens
NTFLS

SEQ ID NO: 158 - protein, 5 aa, Homo sapiens
PLGVE

SEQ ID NO: 159 - protein, 7 aa, Homo sapiens
QPVDLVP

SEQ ID NO: 160 - protein, 5 aa, Homo sapiens
RELGV

SEQ ID NO: 161 - protein, 5 aa, Homo sapiens
RIKIL

SEQ ID NO: 162 - protein, 5 aa, Homo sapiens
RLVPG

SEQ ID NO: 163 - protein, 5 aa, Homo sapiens
TSSSK

SEQ ID NO: 164 - protein, 5 aa, Homo sapiens
VALLV

SEQ ID NO: 165 - protein, 5 aa, Homo sapiens
VDLVP

SEQ ID NO: 166 - protein, 5 aa, Homo sapiens
VLGVL

SEQ ID NO: 167 - protein, 5 aa, Homo sapiens
VLVPH

SEQ ID NO: 168 - protein, 6 aa, Homo sapiens
VLVPHV

SEQ ID NO: 169 - protein, 5 aa, Homo sapiens
ALREL

SEQ ID NO: 170 - protein, 5 aa, Homo sapiens
ARLDP

SEQ ID NO: 171 - protein, 5 aa, Homo sapiens
CFNCL

SEQ ID NO: 172 - protein, 5 aa, Homo sapiens
DLQQL

SEQ ID NO: 173 - protein, 5 aa, Homo sapiens
EAVEA

SEQ ID NO: 174 - protein, 5 aa, Homo sapiens
GEVLV

SEQ ID NO: 175 - protein, 5 aa, Homo sapiens
GLPQG

SEQ ID NO: 176 - protein, 5 aa, Homo sapiens
HAAVD

SEQ ID NO: 177 - protein, 5 aa, Homo sapiens
HRRAR

SEQ ID NO: 178 - protein, 5 aa, Homo sapiens
IKNFK

SEQ ID NO: 179 - protein, 5 aa, Homo sapiens
LLLDQ

SEQ ID NO: 180 - protein, 5 aa, Homo sapiens
VEQDD

SEQ ID NO: 181 - protein, 5 aa, Homo sapiens
VVALL

SEQ ID NO: 182 - protein, 5 aa, Homo sapiens
ALVLL

SEQ ID NO: 183 - protein, 5 aa, Homo sapiens
CISTK

SEQ ID NO: 184 - protein, 5 aa, Homo sapiens
DPPEA

SEQ ID NO: 185 - protein, 5 aa, Homo sapiens
LHALV

SEQ ID NO: 186 - protein, 5 aa, Homo sapiens
LQQLR

SEQ ID NO: 187 - protein, 5 aa, Homo sapiens
PEAEV

SEQ ID NO: 188 - protein, 5 aa, Homo sapiens
PPEAE

SEQ ID NO: 189 - protein, 5 aa, Homo sapiens
RLDPP

SEQ ID NO: 190 - protein, 5 aa, Homo sapiens
SIYNL

SEQ ID NO: 191 - protein, 6 aa, Homo sapiens
SSFTIK

SEQ ID NO: 192 - protein, 5 aa, Homo sapiens
SSYII

SEQ ID NO: 193 - protein, 5 aa, Homo sapiens
VAAGL

SEQ ID NO: 194 - protein, 5 aa, Homo sapiens
VEAEV

SEQ ID NO: 195 - protein, 7 aa, Homo sapiens
CSKCRSK

SEQ ID NO: 196 - protein, 5 aa, Homo sapiens
FSRIL

SEQ ID NO: 197 - protein, 5 aa, Homo sapiens
KRFKK

SEQ ID NO: 198 - protein, 5 aa, Homo sapiens
LRIIT

SEQ ID NO: 199 - protein, 5 aa, Homo sapiens
NITSF

SEQ ID NO: 200 - protein, 8 aa, Homo sapiens
VLLLLVAV

SEQ ID NO: 201 - protein, 9 aa, Homo sapiens
LLPAADLDD

SEQ ID NO: 202 - protein, 6 aa, Homo sapiens
GAGAAL

SEQ ID NO: 203 - protein, 6 aa, Homo sapiens
LGVLVP

SEQ ID NO: 204 - protein, 7 aa, Homo sapiens
LVLLPLV

SEQ ID NO: 205 - protein, 6 aa, Homo sapiens
LLGVGG

SEQ ID NO: 206 - protein, 8 aa, Homo sapiens
GVLPQLEQ

SEQ ID NO: 207 - protein, 9 aa, Homo sapiens
AALGVLVPL